(19) United States
(12) Patent Application Publication
(10) Pub. No.: US 2017/0183376 A1
(43) Pub. Date: Jun. 29, 2017

(54) METHODS OF PURIFYING ANTIBODIES

(71) Applicant: InSight Biopharmaceuticals Ltd., Rehovot (IL)

(72) Inventors: Ronny PINKUS, RaAnana (IL); Meir BUTBUL, Rehovot (IL)

(21) Appl. No.: 15/314,081

(22) PCT Filed: Jun. 24, 2015
(86) PCT No.: PCT/IL2015/050645

§ 371 Date: Nov. 27, 2016

Related U.S. Application Data
(60) Provisional application No. 62/016,153, filed on Jun. 24, 2014.

(51) Int. Cl. C07K 1/36; C07K 16/24; C07K 1/16; C07K 1/22; C07K 1/18

(52) U.S. Cl. CPC C07K 1/36; C07K 1/22; C07K 1/18; C07K 1/165; C07K 16/241; C07K 2317/21; C07K 2317/24

(57) ABSTRACT

Methods of purifying an antibody (Ab) from a mixture comprising impurities are disclosed. One method comprises:
(a) purifying the Ab using an affinity resin;
(b) purifying the Ab using a cation exchange (CEX) resin; and
(c) purifying the Ab using a mixed mode (MM) resin which comprises anion exchange and hydrophobic interaction functional groups; wherein step (b) follows step (a) and step (c) follows step (b), and wherein the method does not comprise use of a hydrophobic interaction chromatography (HIC) medium, thereby purifying the Ab.

### FIELD AND BACKGROUND OF THE INVENTION

The present invention, in some embodiments thereof, relates to a method of purifying an antibody and, more particularly, but not exclusively, to an anti-TNFα antibody.

Large-scale purification of proteins remains a significant challenge in the biopharmaceutical industry as efficient and cost-effective methods are required to achieve desired yields and purity levels. Therapeutic proteins are primarily products of recombinant DNA technology, i.e., cloning and expression of a heterologous gene in prokaryotic or eukaryotic systems. However, proteins expressed by recombinant DNA methods are typically associated with impurities such as host cell proteins (HCP), host cell DNA (HCD), viruses, etc. Also, there is significant heterogeneity in the expression of the desired protein, in the form of charged variants (typically acidic, lower pI variants and basic, higher pI variants). Further, multimeric proteins, such as antibodies, have a higher tendency to aggregate, contributing to significantly increased impurity levels.

The presence of these impurities, including aggregates and undesirable charged variants, is a potential health risk, and hence their removal from a final product is a regulatory requirement. Thus drug regulatory agencies such as United States Food and Drug Administration (FDA) require that biopharmaceuticals be free from impurities, both product related (aggregates or degradation products) and process related (media components, HCP, DNA, chromatographic media used in purification, endotoxins, viruses, etc). Thus, elimination of impurities from a final product is mandatory and poses a significant challenge in the development of methods for the purification of therapeutic proteins.

Antibodies constitute one of the most important classes of therapeutic proteins, especially in the areas of oncology, arthritis and other chronic diseases. Various patent applications teach the purification of antibodies including WO2007/117490; WO2010/141039; WO2013/158279; WO2013/176754; WO2013/066707 and WO2011/015919.

However there is still an unmet need for alternative chromatography processes which are capable of separating the antibody from aggregates and complexes which may form as a result of process-driven modifications or manufacturing conditions.

### SUMMARY OF THE INVENTION

According to an aspect of some embodiments of the present invention there is provided a method of purifying an antibody (Ab) from a mixture comprising impurities comprising:

(a) purifying the Ab using an affinity resin;

(b) purifying the Ab using a cation exchange (CEX) resin; and

(c) purifying the Ab using a mixed mode (MM) resin which comprises anion exchange and hydrophobic interaction functional groups; wherein step (b) follows step (a) and step (c) follows step (b), and wherein the method does not comprise use of a hydrophobic interaction chromatography (HIC) medium, thereby purifying the Ab.

According to an aspect of some embodiments of the present invention there is provided a method of purifying an antibody (Ab) from a mixture comprising impurities comprising:

(a) purifying the Ab using an affinity resin;

(b) purifying the Ab using a mixed mode (MM) resin which comprises anion exchange and hydrophobic interaction functional groups;

(c) purifying the Ab using a CEX membrane adsorber, wherein the method does not comprise use of a hydrophobic interaction chromatography (HIC) medium.

According to an aspect of some embodiments of the present invention there is provided a method of purifying an antibody (Ab) from a mixture which comprises impurities comprising:

(a) contacting a loading buffer which comprises the mixture with a chromatography medium, the chromatography medium having been previously equilibrated in an equilibration buffer which is different to the loading buffer; and

(b) eluting the antibody so as to produce an antibody preparation, the antibody preparation having a reduced amount of impurities as compared with the mixture, thereby purifying the antibody.

According to some embodiments of the invention, the affinity resin comprises a protein A resin.

According to some embodiments of the invention, the method does not comprise contacting the Ab with an anion exchange (AEX) chromatography medium.

According to some embodiments of the invention, the at least one of the affinity resin, the CEX resin or the MM resin is formed into a column.

According to some embodiments of the invention, the Ab is a recombinant Ab.

According to some embodiments of the invention, the Ab is monoclonal Ab (mAb).

According to some embodiments of the invention, the Ab is an antibody to tumor necrosis factor (TNF).

According to some embodiments of the invention, the Protein A resin comprises mAbSelect SuRe™.

According to some embodiments of the invention, the method further comprises performing a viral inactivation step following step (a) and prior to step (b).

According to some embodiments of the invention, the viral inactivation step is effected by lowering the pH of the first eluate to a pH between 3 and 4.

According to some embodiments of the invention, the method further comprises performing a filtration step following the viral inactivation step and prior to step (b).

According to some embodiments of the invention, the CEX resin comprises a SO₃ functional group.

According to some embodiments of the invention, the CEX resin comprises Eshmuno-S™ resin.

According to some embodiments of the invention, the CEX membrane comprises Sartobind S™.

According to some embodiments of the invention, the mixed mode resin is Capto Adhere™ resin.

According to some embodiments of the invention, the method further comprises filtering the Ab following step (c).

According to some embodiments of the invention, the filtering is selected from the group consisting of a depth filtration step, a nanofiltration step, an ultrafiltration step, an absolute filtration step and combination thereof.

According to some embodiments of the invention, the mixture is loaded onto the affinity resin at a loading concentration of 20-100 mg of antibody per ml of the resin.

According to some embodiments of the invention, the step (a) comprises eluting the Ab from the affinity resin using a buffer having a pH of about 3.2-3.8.

According to some embodiments of the invention, the Ab is loaded on to the CEX resin in a loading buffer comprising acetate ions.

According to some embodiments of the invention, the CEX buffer is equilibrated with an equilibration buffer comprising citrate ions.

According to some embodiments of the invention, the equilibration buffer is devoid of acetate ions.

According to some embodiments of the invention, the Ab is eluted from the CEX buffer using a citrate buffer.

According to some embodiments of the invention, the step (b) comprises washing the CEX resin with a citrate buffer and a HEPES buffer prior to eluting.

According to some embodiments of the invention, the Ab is loaded onto the CEX resin in a loading buffer comprising 30-50 mg of antibody per ml of the resin.

According to some embodiments of the invention, the Ab is loaded onto the MM resin in a loading buffer comprising 2-9 mg of protein per ml of the resin.

According to some embodiments of the invention, the MM resin is equilibrated with an equilibration buffer comprising phosphate ions.

According to some embodiments of the invention, the equilibration buffer is devoid of citrate ions.

According to some embodiments of the invention, the Ab is contacted with the MM resin in a loading buffer comprising phosphate ions.

According to some embodiments of the invention, the loading buffer further comprises citrate ions.

According to some embodiments of the invention, the method further comprises performing a viral inactivation step following step (c).

According to some embodiments of the invention, the viral inactivation step is effected by lowering the pH of the mixture to a pH between 3 and 4.

According to some embodiments of the invention, the antibody is expressed in CHO cells.

According to some embodiments of the invention, the antibody is a human antibody, humanized antibody, a chimeric antibody, or a multivalent antibody, or an antigen-binding portion thereof.

According to some embodiments of the invention, the chromatography medium is selected from the group consisting of anion exchange (AEX) resin, CEX resin and MM resin.

According to some embodiments of the invention, the chromatography medium comprises CEX resin and/or MM resin.

According to some embodiments of the invention, the Ab comprises an anti-TNF antibody.

According to some embodiments of the invention, the loading buffer for the CEX resin comprises acetate ions and an equilibration buffer for the CEX resin comprises citrate ions.

According to some embodiments of the invention, the equilibration buffer for the CEX resin is devoid of acetate ions.

According to some embodiments of the invention, the loading buffer for the MM resin comprises citrate ions and an equilibration buffer for the MM resin comprises phosphate ions.

According to some embodiments of the invention, the equilibration buffer is devoid of citrate ions.

According to some embodiments of the invention, the loading buffer further comprises citrate ions.

According to some embodiments of the invention, the chromatography medium does not comprise hydrophobic interaction chromatography (HIC) medium.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a chromatogram of adalimumab purification by affinity chromatography on a MabSelect SuRe™. Clarified harvest was loaded on a GE Healthcare XK 50/30 column packed with 490 ml resin MabSelect SuRe™ (GE Healthcare; 5 cm I.D.×19 cm L) at a residence time NLT 6 min in a ‘down-flow’ direction. Before loading (Load), the column was equilibrated with Equilibration buffer, which has the same properties as the clarified harvest e.g. pH, conductivity. After loading, the column was washed with Equilibration buffer until the absorbance at 280 nm returned to baseline and additional 8 CV of Equilibration buffer. Adalimumab was eluted with 100 mM acetate buffer pH 3.6, at a flow rate of 80 ml/minute (Elution).

FIG. 2 is a chromatogram of mAb purification by Cation Exchange Chromatography on Eshmuno-S. The elution fraction from MabSelect SuRe column is acidified to pH 3.8, after 30 min at room temperature, the sample is neutralized to pH 4.5 and then diluted to a conductivity of 3.5 mS. This sample is loaded on to a column packed with Eshmuno-S (Merck) resin (4.4 cm I.D.×20 cm L) after 0.45 μm filtration. Before loading, the column is washed with 20 mM citrate buffer pH 5.2 (Equilibration buffer). After loading, the column is washed with the same buffer (Unbound wash), and then with HEPES 10 mM pH 6.8 until stable pH and conductivity are reached (Wash 1). The column is then washed with 10 CV of 10 mM HEPES pH 8.0 (Wash2) and then with HEPES 10 mM pH 6.8 until stable pH and conductivity are reached (Wash 3). The column is then equilibrated with Equilibration buffer. Adalimumab is eluted with a linear gradient of 20 CV from 20 mM citrate buffer pH 5.2 to 100% 0.4 M NaCl in 20 mM citrate buffer pH 5.2 at a flow rate of 40 ml/minute. Peak collection starts when the O.D reaches 200 mAU and ends when the absorbance is reduced to a third of its peak maximum. The chromatogram shows the absorbance at 254 nm (blue line) and % of buffer B (0.4 M NaCl in 20 mM citrate buffer pH 5.2, green line). The conductivity and pH are presented in brown and grey respectively.

FIG. 3 illustrates the purification of adalimumab by Mixed-mode Chromatography on a Capto-Adhere Column, Flow-Through Mode. Before loading, adalimumab purified from Eshmuno-S column is diluted with phosphate buffer to a pH higher than 7.0. The sample is loaded at 230 cm/h after equilibration of the column (2.6 cm I.D*20 cm L) with a buffer that has the same properties as the sample e.g. pH, conductivity. Collection of adalimumab starts when absorbance at 280 nm reaches ˜10 mAU (fr1). After loading, the column is washed with equilibration buffer. Collection ends when the absorbance at 280 nm had decreased to 200 mAU (fr2). The chromatogram shows the absorbance at 280 nm profile (blue line), pH (grey line), and conductivity (brown line).

FIG. 4 illustrates isoelectric focusing/GelCode® Blue staining for adalimumab generated as described herein and Humira. Samples of 9 μg adalimumab and Humira were separated on a FocusGel IEF 6-11 polyacrylamide gel, using Multiphor II Electrophoresis unit, stained with GelCode® Blue Stain reagent.

FIGS. 5A-B are flow diagrams summarizing the method of isolating antibodies according to embodiments of the present invention.

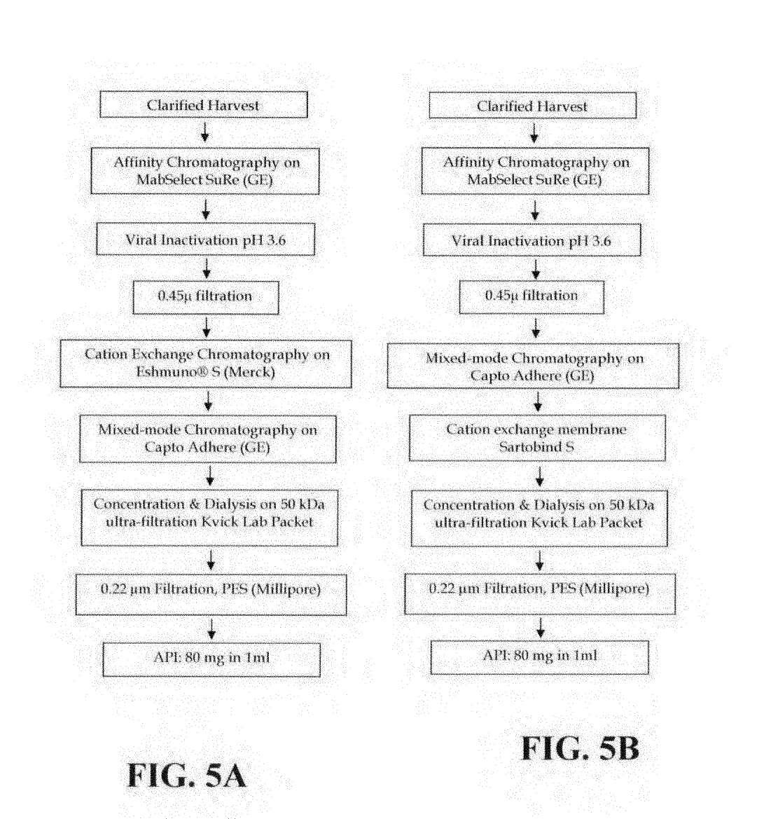

FIGS. 6A-B. A. Adalimumab purified as described herein, purity by size exclusion chromatography (SEC-HPLC) on a ACQUITY BEH200 UPLC, Waters, column. B. “Zoom-in” chromatogram of Adalimumab API.

FIG. 7. Purification of infliximab by affinity chromatography on a MabSelect SuRe™ Column. Chromatogram of infliximab purification by affinity chromatography on a MabSelect SuRe™ column is shown. Clarified infliximab, was loaded (Start Load to End Load) on a GE Healthcare XK 26/20 column packed with 64 ml resin MabSelect SuRe™ (GE Healthcare; 2.6 cm I.D.×12 cm L) at a flow rate of ≦5.6 ml/minute in a ‘down-flow’ direction. Before loading, the column was equilibrated with 5 CV of 20 mM phosphate buffer, 150 mM NaCl, pH 7.4. After loading, the column was washed with 20 mM phosphate buffer 150 mM, NaCl, pH 7.4, until the absorbance at 280 nm returned to baseline and additional 2 CV of 20 mM phosphate buffer, 150 mM NaCl, pH 7.4. The column was washed again with 20 mM phosphate buffer, 1 M NaCl, 0.05% triton x 100, pH 7.4 (Wash 1) followed by washing with 100 mM acetate buffer pH 5.0, at a flow rate of 5.6 ml/minute (Wash 2), until the absorbance at 280 nm, pH and conductivity returns to a low and stable reading. InSight-Infliximab was eluted with 100 mM acetate buffer pH 3.6, at a flow rate of 5.6 ml/minute (Start-Elution to End Elution).

FIG. 8 illustrates purification of infliximab by cation-exchange chromatography on an Eshmuno-S column. Chromatogram of infliximab purification by cation exchange chromatography on Eshmuno-S column is shown. Infliximab after the viral inactivation steps was diluted with water to conductivity of 3.5 mS and loaded on a Millipore column packed with 44 ml (1.6 cm I.D.×22 cm L) Eshmuno-S (Merck) resin(Load). Before loading the column was washed with 3 CV of water followed by 5 CV of 5 mM phosphate buffer, 1 M sodium chloride pH 7.2 at a flow rate of 5 ml/minute at room temperature and then equilibrated with 10 CV of 20 mM acetate buffer pH 5.8. After loading, the column was washed with 3 CV of 20 mM Acetate buffer pH 5.8 and 12 CV of 5 mM phosphate buffer pH 7.2 (Wash 1 and 2 respectively). InSight-Infliximab was eluted with a linear gradient of 40 CV to 100% of 0.3 M sodium chloride in 10 mM phosphate buffer pH 6.0 at a flow rate of 5 ml/minute. Peak collection started when the absorbance of UV254 reached 10 U (Start-Elution) and finished when the absorbance reduced to third of its peak maximum (End Elution).

FIG. 9 illustrates the purification of InSight-Infliximab by Mixed-mode chromatography on a Capto-Adhere Column, Flow-Through mode. Chromatogram of InSight-Infliximab purification by mixed-mode chromatography on a Capto-Adhere column is shown. Before loading (Load), a GE Healthcare XK 16/20 column, packed with ˜22 ml resin (1.6 cm I.D.×11 cm L) was washed with 3 CV of water followed by washing with 5 CV 50 mM phosphate buffer, 1 M sodium chloride, pH 7.0 at a flow rate of 7.7 ml/minute at room temperature (22-25° C.). The column was equilibrated with 5 CV of 50 mM phosphate buffer, pH 7.0. 1320 g InSight-Infliximab after Eshmuno-S column, diluted with 1/10 volume of 500 mM phosphate buffer, was loaded at a flow rate of 7.7 ml/minute. The collection of InSight-Infliximab was started during the flow of the unbound fraction when absorbance at 280 nm reached ˜50 mAU (Start collect). After loading, the column was washed with 2 CV of 50 mM phosphate buffer, pH 7.0, followed by washing with 50 mM phosphate buffer, pH 6.1. (Start pH 6.1). InSight-Infliximab collection was ended when the absorbance at 280 nm had decreased to 300 mAU (End collect).

FIG. 10 illustrates non-reducing SDS-PAGE/GelCode Blue® staining, “In-process control” Gel for InSight-Infliximab purification on a MabSelect SuRe column. Different fractions of InSight-Infliximab intermediates obtained from the affinity chromatography on a MabSelect SuRe column were separated under non-reducing conditions on a 10% tris-glycine Gel (Life technologies) followed by GelCode Blue® (Thermo Scientific) staining. Samples were prepared without DTT; with 10 mM iodoacetamide (non-reducing conditions). Loading fraction of MabSelect SuRe column, “Clarified InSight-Infliximab crude harvest”, (10 μg, harvest); Washing with 20 mM phosphate buffer, 1 M NaCl, 0.05% triton x 100, pH 7.4 fraction of MabSelect SuRe column, (14 μl, Wash 1); Washing 100 mM acetate buffer pH 5.0 fraction of MabSelect SuRe column, (14 μl, Wash 2), Remicade (10 μg, Remicade); Molecular weight pre-stained protein size markers (SeeBlue Plus® 2, Life technologies, 10 μl, Marker). MW in kDa are indicated. Arrows indicate Infliximab bands.

FIG. 11 illustrates the non-reducing SDS-PAGE/GelCode Blue® staining, “In-process controls” for InSight-Infliximab purification: from cation-exchange chromatography to ultrafiltration. Different fractions of InSight-Infliximab intermediates from the cation exchange chromatography on an Eshmuno-S column; mixed mode chromatography on a Capto Adhere column; and samples from the followed ultrafiltration step were separated under non-reducing conditions on a 10% tris-glycine gel (Life Technologies) followed by GelCode Blue® (Thermo Scientific) staining. Samples were prepared without DTT, with 10 mM iodoacetamide (non-reducing conditions). The positions of the samples on the gel from left to right are listed below: Viral inactivated fraction/Eshmuno load (10 μg, Eshmuno load); Elution front-fraction of cation exchange chromatography Eshmuno-S column (14 μl, Eshmuno Pre-peak); Elution fraction of cation exchange chromatography Eshmuno-S column (10 μg, Eshmuno elution); Unbound plus eluted fraction of Capto Adhere column (10 μg, Capto Elution); Retentate fraction of 50 kDa ultrafiltration (10 μg, UF retentate); Remicade (10 μg, Remicade); Molecular weight pre-stained protein size markers (SeeBlue Plus® 2, Life Technologies, 10 μl, Marker), MW in kDa are indicated. Arrows indicate Infliximab bands.

FIG. 12 illustrates isoelectric focusing/GelCode® Blue staining for InSight-Infliximab and Remicade. Samples of 4 μg InSight-Infliximab and Remicade were separated on a FocusGel IEF 6-11 polyacrylamide gel, using Multiphor II electrophoresis unit, stained with GelCode® Blue stain reagent.

### DESCRIPTION OF SPECIFIC EMBODIMENTS OF THE INVENTION

The present invention, in some embodiments thereof, relates to a method of purifying an antibody and, more particularly, but not exclusively, to an anti-TNF antibody.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The purification methods described herein produce highly purified antibodies. The methods result in effective separation of charged variants, as well as removal of impurities such as HCP, aggregates and Protein A leachates and further result in an optimum yield of the desired antibody. Using only three chromatographic media, (protein A affinity resin, cation exchange resin and a mixed mode resin), the conditions described herein result in anti-TNF antibodies which are purified to a level containing less than 0.06% aggregates and less than 1 ppm HCP.

Thus, according to one aspect of the present invention there is provided a method of purifying an antibody (Ab) from a mixture comprising impurities comprising:

(a) purifying the Ab using an affinity resin;

(b) purifying the Ab using a cation exchange (CEX) resin; and

(c) purifying the Ab using a mixed mode (MM) resin which comprises anion exchange and hydrophobic interaction functional groups; wherein step (b) follows step (a) and step (c) follows step (b), and wherein the method does not comprise use of a hydrophobic interaction chromatography (HIC) medium, thereby purifying the Ab.

The term “antibody” refers to an immunoglobulin molecule which comprises four polypeptide chains, two heavy (H) chains and two light (L) chains inter-connected by disulfide bonds. Each heavy chain is comprised of a heavy chain variable region (abbreviated herein as HCVR or VH) and a heavy chain constant region (CH). The heavy chain constant region is comprised of three domains, CHI, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

According to one embodiment, the antibody is not an antibody fragment comprised solely of the antigen binding portion, but also comprises an Fc region. Thus, for example the antibody does not consist solely of (i) a Fab fragment, a monovalent fragment comprising the VL, VH, CL and CHI domains; (ii) a F(ab′)2 fragment, a bivalent fragment comprising two Fab fragments linked by a disulfide bridge at the hinge region; (iii) a Fd fragment comprising the VH and CHI domains; (iv) a Fv fragment comprising the VL and VH domains of a single arm of an antibody, (v) a dAb fragment which comprises a VH domain; or (vi) an isolated complementarity determining region (CDR).

The antibody may be monospecific, (i.e. recognize a single antigen) or bispecific (each arm of the antibody recognizing a different antigen).

The antibody may be of any class e.g. IgAi, IgA₂, IgD, IgE, IgG₁, IgG₂, IgG₃, IgG₄, and IgM antibodies, although preferably the Ab is one which binds to Protein A.

Preferably the antibody is in the class IgG—e.g. IgG₁κ.

The antibody is not limited by any particular method of production. For example, the term antibody includes, without limitation, recombinant antibodies, monoclonal antibodies, and polyclonal antibodies. The antibody employed in the present invention may be any class or subclass of antibody. Furthermore, it may be employed irrespective of the purity of the purification starting materials. Examples include natural human antibodies, humanized and human-type antibodies prepared by genetic recombination, monoclonal antibodies of mice. Humanized and human-type monoclonal antibodies are the most useful from an industrial perspective.

According to a particular embodiment, the antibody is a recombinant, monoclonal antibody.

Methods of producing polyclonal and monoclonal antibodies are well known in the art (See for example, Harlow and Lane, Antibodies: A Laboratory Manual, Cold Spring Harbor Laboratory, New York, 1988, incorporated herein by reference).

Antibodies may be produced by immunization of a non-human animal, preferably a mouse, with an immunogen comprising a desired antigen or immunogen. Alternatively, antibodies may be provided by selection of combinatorial libraries of immunoglobulins, as disclosed for instance in Ward et al (Nature 341 (1989) 544). According to a particular embodiment, the antibody is generated in vitro (i.e. not by injecting the antigen into a living animal).

The step of immunizing a non-human mammal with an antigen may be carried out in any manner well known in the art for stimulating the production of antibodies in a mouse (see, for example, E. Harlow and D. Lane, Antibodies: A Laboratory Manual., Cold Spring Harbor Laboratory Press, Cold Spring Harbor, N.Y. (1988)). In a preferred embodiment, the non-human animal is a mammal, such as a rodent (e.g., mouse, rat, etc.), bovine, porcine, horse, rabbit, goat, sheep, etc. As mentioned, the non-human mammal may be genetically modified or engineered to produce “human” antibodies. Typically, the immunogen is suspended or dissolved in a buffer, optionally with an adjuvant, such as complete Freund's adjuvant. Methods for determining the amount of immunogen, types of buffers and amounts of adjuvant are well known to those of skill in the art and are not limiting in any way on the present invention. These parameters may be different for different immunogens, but are easily elucidated.

Similarly, the location and frequency of immunization sufficient to stimulate the production of antibodies is also well known in the art. In a typical immunization protocol, the non-human animals are injected intraperitoneally with antigen on day 1 and again about a week later. This is followed by recall injections of the antigen around day 20, optionally with adjuvant such as incomplete Freund's adjuvant. The recall injections are performed intravenously or intraperitoneally and may be repeated for several consecutive days. This is followed by a booster injection at day 40, either intravenously or intraperitoneally, typically without adjuvant. This protocol results in the production of antigen-specific antibody-producing B cells after about 40 days. Other protocols may also be utilized as long as they result in the production of B cells expressing an antibody directed to the antigen used in immunization.

In an alternate embodiment, lymphocytes from a non-immunized non-human mammal are isolated, grown in vitro, and then exposed to the immunogen in cell culture. The lymphocytes are then harvested and the fusion step described below is carried out.

For monoclonal antibodies, the next step is the isolation of splenocytes from the immunized non-human mammal and the subsequent fusion of those splenocytes with an immortalized cell in order to form an antibody-producing hybridoma. The isolation of splenocytes from a non-human mammal is well-known in the art and typically involves removing the spleen from an anesthetized non-human mammal, cutting it into small pieces and squeezing the splenocytes from the splenic capsule and through a nylon mesh of a cell strainer into an appropriate buffer so as to produce a single cell suspension. The cells are washed, centrifuged and re-suspended in a buffer that lyses any red blood cells. The solution is again centrifuged and remaining lymphocytes in the pellet are finally re-suspended in fresh buffer.

Once isolated and present in single cell suspension, the lymphocytes are fused to an immortal cell line. This is typically a mouse myeloma cell line, although many other immortal cell lines useful for creating hybridomas are known in the art.

Preferred murine myeloma lines include, but are not limited to, those derived from MOPC-21 and MPC-11 mouse tumors available from the Salk Institute Cell Distribution Center, San Diego, Calif. U.S.A., X63 Ag8653 and SP-2 cells available from the American Type Culture Collection, Rockville, Md. U.S.A. The fusion is effected using polyethylene glycol or the like. The resulting hybridomas are then grown in selective media that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

The hybridomas are typically grown on a feeder layer of macrophages. The macrophages are preferably from littermates of the non-human mammal used to isolate splenocytes and are typically primed with incomplete Freund's adjuvant or the like several days before plating the hybridomas. Fusion methods are described in (Goding, “Monoclonal Antibodies: Principles and Practice,” pp. 59-103, Academic Press, 1986).

The cells are allowed to grow in the selection media for sufficient time for colony formation and antibody production. This is usually between 7 and 14 days. The hybridoma colonies are then assayed for the production of antibodies that bind the immunogen/antigen. The assay is typically a colorimetric ELISA-type assay, although any assay may be employed that can be adapted to the wells that the hybridomas are grown in. Other assays include immunoprecipitation and radioimmunoassay. The wells positive for the desired antibody production are examined to determine if one or more distinct colonies are present. If more than one colony is present, the cells may be recloned and grown to ensure that only a single cell has given rise to the colony producing the desired antibody. Positive wells with a single apparent colony are typically recloned and re-assayed to insure only one monoclonal antibody is being detected and produced.

Hybridomas that are confirmed to be producing a monoclonal antibody are then grown up in larger amounts in an appropriate medium, such as DMEM or RPMI-1640. Alternatively, the hybridoma cells can be grown in vivo as ascites tumors in an animal.

After sufficient growth to produce the desired monoclonal antibody, the growth media containing monoclonal antibody (or the ascites fluid) is separated away from the cells and the monoclonal antibody present therein is purified. DNA encoding the heavy and light chains of the antibody may be readily isolated and sequenced using conventional procedures (e.g., by using oligonucleotide probes that are capable of binding specifically to genes encoding the heavy and light chains of antibodies such as murine or human). Once isolated, the DNA can be ligated into expression vectors, which are then transfected into host cells.

The antibodies according to the invention are typically produced by recombinant means.

To express a recombinant antibody of the invention, DNAs encoding partial or full-length light and heavy chains are inserted into one or more expression vector such that the genes are operatively linked to transcriptional and translational control sequences. (See, e.g., U.S. Pat. No. 6,914,128, the entire teaching of which is incorporated herein by reference). In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into a separate vector or, more typically, both genes are inserted into the same expression vector. The antibody genes are inserted into an expression vector by standard methods (e.g., ligation of complementary restriction sites on the antibody gene fragment and vector, or blunt end ligation if no restriction sites are present). Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein). In addition to the antibody chain genes, a recombinant expression vector of the invention can carry one or more regulatory sequence that controls the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, e.g., in Goeddel; Gene Expression Technology: Methods in Enzymology 185, Academic Press, San Diego, Calif. (1990), the entire teaching of which is incorporated herein by reference. It will be appreciated by those skilled in the art that the design of the expression vector, including the selection of regulatory sequences may depend on such factors as the choice of the host cell to be transformed, the level of expression of protein desired, etc. Suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see, e.g., U.S. Pat. No. 5,168,062 by Stinski, U.S. Pat. No. 4,510,245 by Bell et al. and U.S. Pat. No. 4,968,615 by Schaffher et al., the entire teachings of which are incorporated herein by reference. In addition to the antibody chain genes and regulatory sequences, a recombinant expression vector of the invention may carry one or more additional sequences, such as a sequence that regulates replication of the vector in host cells (e.g., origins of replication) and/or a selectable marker gene. The selectable marker gene facilitates selection of host cells into which the vector has been introduced (see e.g., U.S. Pat. Nos. 4,399,216, 4,634,665 and 5,179,017, all by Axel et al, the entire teachings of which are incorporated herein by reference). For example, typically the selectable marker gene confers resistance to drugs, such as G418, hygromycin or methotrexate, on a host cell into which the vector has been introduced. Suitable selectable marker genes include the dihydrofolate reductase (DHFR) gene (for use in dhfr-host cells with methotrexate selection/amplification) and the neo gene (for G418 selection).

To express an antibody recombinantly, a host cell is transfected with one or more recombinant expression vector carrying DNA fragments encoding the immunoglobulin light and heavy chains of the antibody such that the light and heavy chains are expressed in the host cell and secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel et al. (eds.) Current Protocols in Molecular Biology, Greene Publishing Associates, (1989) and in U.S. Pat. Nos. 4,816,397 & 6,914,128, the entire teachings of which are incorporated herein.

For expression of the light and heavy chains, the expression vector(s) encoding the heavy and light chains is (are) transfected into a host cell by standard techniques. The various forms of the term “transfection” are intended to encompass a wide variety of techniques commonly used for the introduction of exogenous DNA into aprokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, such as mammalian host cells, is suitable because such eukaryotic cells, and in particular mammalian cells, are more likely than prokaryotic cells to assemble and secrete a properly folded and immunologically active antibody.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, e.g., Enterobacteriaceae such as Escherichia, e.g., E. coli, Enterobacter, Erwinia, Klebsiella, Proteus, Salmonella, e.g., Salmonella typhimurium, Serratia, e.g., Serratia marcescens, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One suitable E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting. In addition to prokaryotes, eukaryotic microbes such as filamentous fungi or yeast are suitable cloning or expression hosts for polypeptide encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24, 178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fraitfly), and Bombyx mori have been identified. A variety of viral strains for transfection are publicly available, e.g., the L-I variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

Suitable mammalian host cells for expressing the recombinant antibodies of the invention include Chinese Hamster Ovary (CHO cells) (including dhfr-CHO cells, described in Urlaub and Chasin, (1980) PNAS USA 77:4216-4220, used with a DHFR selectable marker, e.g., as described in Kaufman and Sharp (1982) Mol. Biol. 159:601-621, the entire teachings of which are incorporated herein by reference), NSO myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Other examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse Sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N. Y. Acad. Sci. 383:44-68 (1982)); MRC 5 cells; FS4 cells; and a human hepatoma line (Hep G2), the entire teachings of which are incorporated herein by reference.

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce an antibody may be cultured in a variety of media. Commercially available media such as Ham's F 10™ (Sigma), Minimal Essential Medium™ ((MEM), (Sigma), RPMI-1640 (Sigma), and Dulbecco's Modified Eagle's Medium™ ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells, the entire teachings of which are incorporated herein by reference. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In certain embodiments it may be desirable to transfect a host cell with DNA encoding either the light chain or the heavy chain (but not both) of an antibody of this invention. Thus, for example, the light chain and heavy chain may be expressed in inclusion bodies in bacterial cultures and subsequently refolded.

In an exemplary system for recombinant expression of an antibody of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

When using recombinant techniques, the antibody can be produced intracellularly, in the periplasmic space, or directly secreted into the medium. If the antibody is produced intracellularly, as a first step, the particulate debris, either host cells or lysed cells (e.g., resulting from homogenization), can be removed, e.g., by centrifugation or ultrafiltration. Where the antibody is secreted into the medium, supernatants from such expression systems can be first concentrated using a commercially available protein concentration filter, e.g., an Amicon™ or Millipore Pellicon™ ultrafiltration unit.

Lysis of the cells may be performed by a variety of methods, including mechanical shear, osmotic shock, or enzymatic treatments. Such disruption releases the entire contents of the cell into the homogenate, and in addition produces subcellular fragments that are difficult to remove due to their small size. These are generally removed by differential centrifugation or by filtration. Where the antibody is secreted, supernatants from such expression systems are generally first concentrated using a commercially available protein concentration filter, e.g., an Amicon™ or Millipore Pellicon™ ultrafiltration unit. Where the antibody is secreted into the medium, the recombinant host cells can also be separated from the cell culture medium, e.g., by tangential flow filtration.

As mentioned herein above, the antibodies of the present invention may be humanized.

Humanized forms of non-human (e.g., murine) antibodies are chimeric molecules of immunoglobulins which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

In one embodiment, the antibody of the invention binds to TNFα—e.g. human TNFα. The phrase “Tumor necrosis factor-α” (abbreviated herein as hTNFα or TNFα) is a multifunctional pro-inflammatory cytokine secreted predominantly by monocytes/macrophages that has effects on lipid metabolism, coagulation, insulin resistance, and endothelial function. TNFα is a soluble homotrimer of 17 kD protein subunits. A membrane-bound 26 kD precursor form of TNFα also exists. It is found in synovial cells and macrophages in tissues. Cells other than monocytes or macrophages also produce TNFα. For example, human non-monocytic tumor cell lines produce TNFα as well as CD4+ and CD8+peripheral blood T lymphocytes and some cultured T and B cell lines produce TNFα. The nucleic acid encoding TNFα is available as GenBank Accession No. X02910 and the polypeptide sequence is available as GenBank Accession No. CAA26669. The term human TNFα is intended to include recombinant human TNFα (rh TNFα), which can be prepared by standard recombinant expression methods.

Examples of anti-TNFα antibodies include, but are not limited to, anti-TNFα human antibodies as well as those described in U.S. Pat. Nos. 6,090,382; 6,258,562; 6,509,015, and in U.S. patent application Ser. Nos. 09/801,185 and 10/302,356, each of which is incorporated by reference herein. In one embodiment, the TNFα antibody is infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor), antibodies described in WO 02/12502, and adalimumab (Humira® Abbott Laboratories, a human anti-TNF Ab, described in U.S. Pat. No. 6,090,382 as D2E7).

As used herein, the term “adalimumab,” refers to a FDA approved fully humanized IgG1, TNF-alpha inhibitor monoclonal antibody (tradename Humira®) produced by Abbott Laboratories. Each IgG antibody molecule comprises two kappa light chains and two human IgG1 heavy chains, the total molecular weight of Adalimumab is 148 kDa. Each light chain consists of 214 amino acid residues and each heavy chain consists of 451 amino acid residues.

It will be appreciated that the antibody may be a biosimilar of the above mentioned antibodies.

As used herein, the term “biosimilar” refers to a biopharmaceutical which is deemed to be comparable in quality, safety, and efficacy to reference product marketed by an innovator company.

The invention also contemplates antibodies which have sequences at least 90%, at least 91%, at least 92%, at least 93%, at least 94%, at least 95%, at least 96%, at least 97%, at least 98%, at least 99%, identical to those disclosed herein for the particular antibodies described herein above. When the antibodies are not 100% identical to those described herein above, it is conceived that they may comprise either conservative or non-conservative amino acid changes.

The antibodies described herein are purified from a mixture comprising impurities.

As used herein, a “mixture” comprises the antibody of interest (for which purification is desired) and one or more contaminant, i.e., impurities. The mixture can be obtained directly from a host cell or organism producing the polypeptide. Without intending to be limiting, examples of mixtures that can be purified according to a method of the present invention include harvested cell culture fluid, cell culture supernatant and conditioned cell culture supernatant.

According to one embodiment, the impurity is a protein aggregate.

As used herein, the term “protein aggregate” refers to multimers (such as dimers, tetramers or higher order aggregates) of the Ab to be purified and may result e.g. in high molecular weight aggregates.

According to a specific embodiment, the mixture comprises a Chinese hamster ovary cell protein, referred to herein as “CHOP”. The amount of CHOP present in a mixture comprising a protein of interest provides a measure of the degree of purity for the protein of interest. Typically, the amount of CHOP in a protein mixture is expressed in parts per million relative to the amount of the protein of interest in the mixture. It is understood that where the host cell is another cell type, e.g., a eukaryotic cell other than CHO cells, an insect cell, or a plant cell, of a yeast cell, host cell protein (HCP) refers to the proteins, other than target protein, found in a lysate of the host cell.

Mixtures can, for example, be aqueous solutions, organic solvent systems, or aqueous/organic solvent mixtures or solutions. The mixtures are often complex mixtures or solutions comprising many biological molecules (such as proteins, antibodies, hormones, and viruses), small molecules (such as salts, sugars, lipids, etc.) and even particulate matter. While a typical mixture of biological origin may begin as an aqueous solution or suspension, it may also contain organic solvents used in earlier separation steps such as solvent precipitations, extractions, and the like. Examples of mixtures that may contain valuable biological substances amenable to the purification by various embodiments the present invention include, but are not limited to a harvested cell culture fluid, a cell culture supernatant, a conditioned cell culture supernatant from a bioreactor, a homogenized cell suspension, plasma, plasma fractions and milk.

Preferably the mixture has not already been subjected to a chromatography step, e.g., non-affinity chromatography, affinity chromatography, etc.

According to one embodiment, the mixture has been clarified.

As used herein, the term “clarified” refers to a sample (i.e. a cell suspension) having undergone a solid-liquid separation step involving one or more of centrifugation, microfiltration and depth filtration to remove host cells and/or cellular debris. A clarified fermentation broth may be a cell culture supernatant. Clarification is sometimes referred to as a primary or initial recovery step and typically occurs prior to any chromatography or a similar step.

Following generation of the antibody, purification steps are taken so as to decrease the level of impurities in the mixture.

According to this aspect of the present invention, three essential chromatography purification steps are taken, which have to occur in the following order:

(a) purifying the Ab using an affinity resin; subsequently

(b) purifying the Ab using a cation exchange (CEX) resin; and subsequently

(c) purifying the Ab using a mixed mode (MM) resin.

An alternative to these three steps is as follows:

(a) purifying the Ab using an affinity resin;

(b) purifying the Ab using a mixed mode (MM) resin which comprises anion exchange and hydrophobic interaction functional groups; and

(c) purifying the Ab using a CEX membrane adsorber.

It will be appreciated that intervening steps may be taken during the purification procedure and also following the purification procedure. Preferably, the intervening steps do not comprise use of additional chromatography media such as anion exchange resin or hydrophobic interaction chromatography resin.

Examples of contemplated intervening steps include viral inactivation and filtration, as further described herein below.

According to a particular embodiment, there is no intervening purification step between the use of the CEX resin and the MM resin or the use of the MM resin and CEX membrane adsorber.

The term “chromatography resin” or “chromatography media” are used interchangeably herein and refer to any kind of solid phase which separates an analyte of interest (e.g., an Fc region containing protein such as an immunoglobulin) from other molecules present in a mixture. Usually, the analyte of interest is separated from other molecules as a result of differences in rates at which the individual molecules of the mixture migrate through a stationary solid phase under the influence of a moving phase, or in bind and elute processes. Non-limiting examples include cation exchange resins, affinity resins and mixed mode resins. The volume of the resin, the length and diameter of the column to be used, as well as the dynamic capacity and flow-rate depend on several parameters such as the volume of fluid to be treated, concentration of protein in the fluid to be subjected to the process of the invention, etc. Determination of these parameters for each step is well within the average skills of the person skilled in the art.

Affinity Resin:

In certain embodiments, the mixture is subjected to affinity chromatography to purify the antibody of interest away from impurities. In certain embodiments the chromatographic material is capable of selectively or specifically binding to the antibody of interest. Non-limiting examples of such chromatographic material include: Protein A, Protein A/G, Protein G and Protein L chromatographic material. In specific embodiments, the affinity chromatography step involves subjecting the primary recovery sample to a column comprising a suitable Protein A resin. Protein A resin is useful for affinity purification and isolation of a variety of antibody isotypes, particularly IgG₁, IgG₂, and IgG₄. Protein A is a bacterial cell wall protein that binds to mammalian IgGs primarily through their Fc regions. In its native state, Protein A has five IgG binding domains as well as other domains of unknown function. There are several commercial sources for Protein A resin including, but not limited to, MabSelect SuRe™, MabSelect SuRe LX, MabSelect, MabSeiect Xtra, rProtein A Sepharose from GE Healthcare, ProSep HC, ProSep Ultra, and ProSep Ultra Plus from EMD Millipore, MapCapture from Life Technologies.

In certain embodiments, the Protein A column can be equilibrated with a suitable buffer prior to sample loading. Such a buffer typically has the same properties as the clarified harvest e.g. pH, conductivity. A non-limiting example of a suitable buffer is a 20 mM phosphate buffer, 150 mM NaCl, pH of about 7.4. Following this equilibration, the sample can be loaded onto the column. Preferably, the load should have a minimum residence time NLT of about 2-30 minutes. According to some embodiments the NLT should be between 2-20 minutes or 10-20 minutes. In certain embodiments, the mixture is loaded onto the affinity resin at a loading concentration of 10-80, 20-60, 30-50 or more preferably between 40-50 mg of antibody per ml of resin.

Following the loading of the column, the column can be washed one or multiple times using, e.g., the equilibrating buffer (e.g. a buffer comprising 20 mM sodium phosphate buffer, 150 mM NaCl). Other washes, including washes employing different buffers, can be employed prior to eluting the column. For example, the column can be washed using one or more column volumes of 20 mM sodium phosphate buffer, 1 M NaCl, 0.05% triton, pH 7-7.4 and/or 100 mM acetate buffer pH 5.0. This wash can optionally be followed by one or more washes using the equilibrating buffer.

The Protein A column can then be eluted using an appropriate elution buffer. A non-limiting example of a suitable elution buffer is an acetic acid buffer, pH of about 3.2-3.6. Suitable conditions are, e.g., 0.1 M acetic acid, pH of about 3.5.

The eluate can be monitored using techniques well known to those skilled in the art. For example, the absorbance at OD₂₈₀ can be followed. Column eluate can be collected starting with an initial deflection of about 0.5 AU to a reading of about 0.5 AU at the tailing edge of the elution peak. The elution fraction(s) of interest can then be prepared for further processing.

An exemplary height of an affinity column (e.g. Mab SelectSure) is between 10-24 cm. An exemplary resident time on an affinity column (e.g. Mab SelectSure) is less than 15 minutes, for example about 10 minutes.

Cation Exchange Resin:

Following affinity chromatography, the antibody is further purified using a cation exchange resin (CEX). In performing the separation, the antibody sample mixture can be contacted with the cation exchange material by using any of a variety of techniques, e.g., using a batch purification technique or a chromatographic technique. For the purification of an antibody, the antibody must have a charge opposite to that of the functional group attached to the ion exchange material, e.g., resin, in order to bind. For example, antibodies, which have an overall positive charge when present in a buffer having a pH below the antibody's pI, will bind well to cation exchange material, which contain negatively charged functional groups. Elution is generally achieved by increasing the ionic strength (i.e., conductivity) of the buffer to compete with the solute for the charged sites of the ion exchange matrix. Changing the pH and thereby altering the charge of the solute is another way to achieve elution of the solute. The change in conductivity or pH may be gradual (gradient elution) or stepwise (step elution). Cationic substituents may be attached to matrices in order to form cationic supports for chromatography. Non-limiting examples of cationic exchange substituents include carboxymethyl (CM), sulfoethyl(SE), sulfopropyl(SP), phosphate(P) and sulfonate(S).

Preferably, the CEX resin comprises a SO₃ functional group.

Cellulose ion exchange resins such as DE23™, DE32™, DE52™, CM-23™, CM-32™, and CM-52™ are available from Whatman Ltd. Maidstone, Kent, U.K. SEPHADEX®-based and cross-linked ion exchangers are also known. For example, DEAE-, QAE-, CM-, and SP-SEPHADEX® and DEAE-, Q-, CM- and S-SEPHAROSE® and SEPHAROSE® Fast Flow are all available from Pharmacia AB. Further, both DEAE and CM derivatized ethylene glycol-methacrylate copolymer such as TOYOPEARL™ DEAE-650S or M and TOYOPEARL™ CM-650S or M are available from Tosoh, Philadelphia, Pa.

Further, both DEAE and CM derivatized ethylene glycol-methacrylate copolymer such as TOYOPEARL™ DEAE-650S or M and TOYOPEARL™ CM-650S or M are available from Tosoh, Philadelphia, Pa., or Nuvia S and U OSphere™ S from BioRad, Hercules, Calif., Eshmuno® S from EMD Millipore, Billerica, Calif.

According to a particular embodiment, the CEX resin comprises Eshmuno® S.

According to another embodiment, the CEX resin comprises Fractogel EMD COO⁻ (M).

According to a particular embodiment, the CEX resin is equilibrated prior to loading with an equilibration buffer which is substantially different to the loading buffer in which the antibody is loaded. The difference may lie in terms of the conductivity of the two buffers, the pH of the two buffers and/or the particular ions present in the two buffers.

Thus, for example, the present inventors contemplate that the antibody is loaded on the CEX resin in a loading buffer comprising acetate ions whereas the CEX resin is equilibrated in a buffer comprising citrate ions and not acetate ions. Typically, using this arrangement, the antibody is eluted from the CEX buffer using an elution buffer which comprises the same ions as the equilibration buffer, in this case the citrate buffer.

According to a particular embodiment, the column is washed prior to equilibration with a high salt buffer—e.g. 20 mM citrate buffer, 1 M sodium chloride pH 7.2 or 5 mM phosphate buffer, 1 M sodium chloride pH 7.2.

The CEX resin may be washed prior to eluting e.g. using a citrate buffer and a HEPES buffer.

According to a particular embodiment, the column is washed with 10 mM HEPES pH 6.8 then with 10 mM HEPES pH 8.0 and again with 10 mM HEPES pH 6.8. The column is equilibrated with 20 mM citrate buffer pH 5.2 and the antibody is then eluted with a gradient of 0.4 M sodium chloride in 20 mM citrate buffer pH 5.2.

Another washing protocol is a first wash with 20 mM acetate buffer pH 5.8, a second wash with 5 mM phosphate buffer pH 7.2 and a third wash with 10 mM phosphate buffer pH 6.0. The antibody may then elute with a gradient of 0 to 100% of 0.3 M sodium chloride in 10 mM phosphate buffer pH 6.0. Still another washing protocol includes a first wash of 5 mM phosphate buffer pH 7.2 and elution with a gradient of 20 or 40 CV from 0 to 100% of 0.1 M sodium chloride in 5 mM phosphate buffer pH 7.2.

The Ab may be loaded onto the CEX resin in a loading buffer comprising 20-40 mg of protein per ml of the resin. Typically, the loading solution is adjusted to a pH of about 4.5 prior to loading.

According to yet another embodiment, the load buffer may undergo buffer exchange to a phosphate buffer (e.g. 5 mM phosphate buffer, pH 7.2). This may be effected for example using 0.1 m² of 50 kDa Omega™ Membrane, Centrasette II, Medium screen (Pall) and a PES 0.8/0.45 μm filter (Pall). In this case the column is typically first equilibrated with 5 mM phosphate buffer pH 7.2 and washed with 5 mM phosphate buffer pH 7.2. Elution may be performed with a gradient of 0 to 100% of 0.1 M sodium chloride in 10 mM phosphate buffer pH 7.2.

The cation exchange procedure can be carried out at or around room temperature.

An exemplary resident time on an affinity column (e.g. Eshmuno S) is less than about 4 minutes.

CEX Membrane Adsorbers:

An ion exchange chromatography membrane will bind a compound with an overall positive or negative charge. Binding sites are located along the pores of the adsorber. The compound is transported to the binding site by flowing. A positively charged membrane (anion exchanger) will bind a compound with an overall negative charge. Conversely, a negatively charged membrane (cation exchanger) will bind a compound with an overall positive charge.

Ion exchange membranes can be further categorized as either strong or weak. Strong ion exchange membranes are charged (ionized) across a wide range of pH levels. Weak ion exchange membranes are ionized within a narrow pH range.

In general, ion exchange membranes have pore sizes of 0.1 to 100 μm. As a reference, Sartobind S (Sartorius AG) is a strong cation exchange membrane having a nominal pore size of 3-5 μm and is commercially available in a single or multiple layer format, and Mustang S (Pall Corporation) is a strong cation exchange membrane having a nominal pore size of 0.8 μm and is similarly commercially available in a single or multiple layer format.

Mixed Mode Chromatography:

Following purification using a CEX media, the antibody is next subjected to mixed mode (MM) purification. Mixed mode chromatography is chromatography that utilizes a mixed mode media, such as, but not limited to Capto Adhere™ available from GE Healthcare. Such a media comprises a mixed mode chromatography ligand. In certain embodiments, such a ligand refers to a ligand that is capable of providing at least two different, but co-operative, sites which interact with the substance to be bound. One of these sites gives an attractive type of charge-charge interaction between the ligand and the substance of interest. The other site typically gives electron acceptor-donor interaction and/or hydrophobic and/or hydrophilic interactions. Electron donor-acceptor interactions include interactions such as hydrogen-bonding, π-π, cation-π, charge transfer, dipole-dipole, induced dipole etc.

In certain embodiments, the mixed mode chromatography media is comprised of mixed mode ligands coupled to an organic or inorganic support, sometimes denoted a base matrix, directly or via a spacer. The support may be in the form of particles, such as essentially spherical particles, a monolith, filter, membrane, surface, capillaries, etc. In certain embodiments, the support is prepared from a native polymer, such as cross-linked carbohydrate material, such as agarose, agar, cellulose, dextran, chitosan, konjac, carrageenan, gellan, alginate etc. To obtain high adsorption capacities, the support can be porous, and ligands are then coupled to the external surfaces as well as to the pore surfaces. Such native polymer supports can be prepared according to standard methods, such as inverse suspension gelation (S Hjerten: Biochim Biophys Acta 79(2), 393-398 (1964). Alternatively, the support can be prepared from a synthetic polymer, such as cross-linked synthetic polymers, e.g. styrene or styrene derivatives, divinylbenzene, acryl amides, acrylate esters, methacrylate esters, vinyl esters, vinyl amides etc. Such synthetic polymers can be produced according to standard methods, see e.g. “Styrene based polymer supports developed by suspension polymerization” (R Arshady: Chimica e L'Industria 70(9), 70-75 (1988)). Porous native or synthetic polymer supports are also available from commercial sources, such as GE healthcare, Uppsala, Sweden.

In certain embodiments, the mixed-mode resin comprises a negatively charged part and a hydrophobic part. In one embodiment, the negatively charged part is an anionic carboxylate group or anionic sulfo group for cation exchange. Examples of such supports include, but are not limited to, Capto Adhere® (GE Healthcare). Capto Adhere® is a strong anion exchanger with multimodal functionality which confers different selectivity to the resin compared to traditional anion exchangers. The Capto Adhere® ligand (N-Benzyl-N-methyl ethanolamine) exhibits multiple modes of protein-interactive chemistries, including ionic interaction, hydrogen bonding and hydrophobic interaction. The multimodal functionality of the resin confers it with an ability to remove antibody dimers and aggregates, leached protein A, host cell proteins (HCP), antibody/HCP complexes, process residuals and viruses. The resin may be used in flow-through mode in the context of a production scale polishing step employing operational parameters designed to have the mAb pass directly through the column while the contaminants are adsorbed (e.g. for Adalimumab).

For the purification of Adalimumab, the MM resin may be equilibrated prior to loading with an equilibration buffer which is substantially different to the loading buffer in which the antibody is loaded. The difference may lie in terms of the conductivity of the two buffers, the pH of the two buffers and/or the particular ions present in the two buffers.

Thus, for example, the present inventors contemplate that the antibody is loaded on the MM resin in a loading buffer comprising citrate ions whereas the MM resin is equilibrated in a buffer comprising phosphate ions and not citrate ions.

According to a particular embodiment, the column is equilibrated with 50 mM phosphate buffer, 166 mM sodium chloride, pH of about 7.8.

The Ab may be loaded onto the MM resin in a loading buffer comprising 2-9 mg of protein per ml of the resin. Typically, the loading solution is adjusted to a pH of about 7.8 or less, and a conductivity of <28 mS/cm² prior to loading.

The MM exchange procedure can be carried out at or around room temperature.

An exemplary height of a mixed mode column (e.g. Capto Adhere) is between 10-20 cm.

As mentioned herein above, the present invention contemplates adding additional steps to the three required steps, so long as the three required steps remain in the specified order.

Thus according to a particular embodiment, the antibody is subjected to a viral inactivation step. This may be performed at any stage during the purification procedure. According to one embodiment, the viral inactivation is effected between the affinity purification step and the CEX purification step or alternatively following the MM step.

The phrase “viral inactivation”, as used herein, refers to a decrease in the activity of adventitious enveloped viruses in a particular sample (“inactivation”). Such decreases in the activity of enveloped viruses can be on the order of about 3 log reduction factor (LRF) preferably of about 4 LRF, more preferably of about 5 LRF, even more preferably of about 6 LRF.

Any one or more of a variety of methods of viral inactivation can be used including heat inactivation (pasteurization), pH inactivation, solvent/detergent treatment, UV and γ-ray irradiation and the addition of certain chemical inactivating agents such as β-propiolactone or e.g., copper phenanthroline as in U.S. Pat. No. 4,534,972, the entire teaching of which is incorporated herein by reference.

Methods of pH viral inactivation include, but are not limited to, incubating the mixture for a period of time at low pH, and subsequently neutralizing the pH. In certain embodiments the mixture will be incubated at a pH of between about 2 and 5, preferably at a pH of between about 3 and 4, and more preferably at a pH of about 3.6. The pH of the sample mixture may be lowered by any suitable acid including, but not limited to, citric acid, acetic acid, caprylic acid, or other suitable acids. The choice of pH level largely depends on the stability profile of the antibody product and buffer components. It is known that the quality of the target antibody during low pH virus inactivation is affected by pH and the duration of the low pH incubation. In certain embodiments the duration of the low pH incubation will be from 0.5 hr to 2 hr, preferably 0.5 hr to 1.5 hr, and more preferably the duration will be about 1 hr. Virus inactivation is dependent on these same parameters in addition to protein concentration, which may limit inactivation at high concentrations. Thus, the proper parameters of protein concentration, pH, and duration of inactivation can be selected to achieve the desired level of viral inactivation.

In certain embodiments viral filtration is performed. This can be achieved via the use of suitable filters. A non-limiting example of a suitable filter is the Ultipor DV50™ filter from Pall Corporation. In certain embodiments, alternative filters are employed for viral inactivation, such as, but not limited to, Sartorius filters, Viresolve™ filters (Millipore, Billerica, Mass.); Zeta Plus VR™ filters (CUNO; Meriden, Conn.); and Planova™ filters (Asahi Kasei Pharma, Planova Division, Buffalo Grove, 111.).

In those embodiments where viral inactivation is employed, the sample mixture can be adjusted, as needed, for further purification steps. For example, following low pH viral inactivation the pH of the sample mixture is typically adjusted to a more neutral pH, e.g., from about 4 to about 8, and preferably about 4.6, prior to continuing the purification process. Additionally, the mixture may be flushed with water for injection (WFI) to obtain a desired conductivity.

Certain embodiments of the present invention employ filtration prior to loading of a sample onto a column. Thus, for example, the present invention contemplates passing the sample over a filter (for example a 0.45 μm filter) prior to loading on to a cation exchange column or a mixed mode column. Examples of such filters include hydrophilic DURAPORE PVDF or PES polyethersulfone filters.

Certain embodiments of the present invention employ ultrafiltration and/or diafiltration steps to further purify and concentrate the antibody sample. Typically, this is carried out following the MM purification step. Ultrafiltration is described in detail in: Microfiltration and Ultrafiltration: Principles and Applications, L. Zeman and A. Zydney (Marcel Dekker, Inc., New York, N.Y., 1996); and in: Ultrafiltration Handbook, Munir Cheryan (Technomic Publishing, 1986; ISBN No. 87762-456-9). A preferred filtration process is Tangential Flow Filtration as described in the Millipore catalogue entitled “Pharmaceutical Process Filtration Catalogue” pp. 177-202 (Bedford, Mass., 1995/96). Ultrafiltration is generally considered to mean filtration using filters with a pore size that allow transfer of protein with average size of 50 kDa (for example) or smaller. By employing filters having such small pore size, the volume of the sample can be reduced through permeation of the sample buffer through the filter while antibodies are retained behind the filter.

Diafiltration is a method of using ultrafilters to remove and exchange salts, sugars, and non-aqueous solvents, to separate free from bound species, to remove low molecular-weight material, and/or to cause the rapid change of ionic and/or pH environments. Microsolutes are removed most efficiently by adding solvent to the solution being ultrafiltered at a rate approximately equal to the ultratfiltration rate. This washes microspecies from the solution at a constant volume, effectively purifying the retained antibody. In certain embodiments of the present invention, a diafiltration step is employed to exchange the various buffers used in connection with the instant invention, optionally prior to further chromatography or other purification steps, as well as to remove impurities from the antibody.

The novel methods of purifying the antibodies described in the present application result in the antibodies themselves also being novel.

Thus according to another aspect of the present an antibody having been purified according to the methods described herein.

Antibodies obtained using the process of the invention may be incorporated into pharmaceutical compositions suitable for administration to a subject. Typically, the pharmaceutical composition comprises an antibody and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it is preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of antibody, or antigen-binding portion thereof.

Pharmaceutical compositions comprising antibodies purified using the methods of the invention may be found in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies or other TNFα inhibitors. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody, or antigen-binding portion thereof) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

Supplementary active compounds can also be incorporated into the compositions. In certain embodiments, an antibody, or antigen-binding portion thereof, for use in the methods of the invention is coformulated with and/or coadministered with one or more additional therapeutic agents. For example, an anti-hTNFα antibody or antibody portion of the invention may be coformulated and/or coadministered with one or more DMARD or one or more NSAID or one or more additional antibodies that bind other targets (e.g., antibodies that bind other cytokines or that bind cell surface molecules), one or more cytokines, soluble TNFα receptor (see e.g., PCT Publication No. WO 94/06476) and/or one or more chemical agents that inhibit hTNFα production or activity (such as cyclohexane-ylidene derivatives as described in PCT Publication No. WO 93/19751) or any combination thereof. Furthermore, one or more antibodies of the invention may be used in combination with two or more of the foregoing therapeutic agents. Such combination therapies may advantageously utilize lower dosages of the administered therapeutic agents, thus avoiding possible side effects, complications or low level of response by the patient associated with the various monotherapies.

In one embodiment, the invention includes pharmaceutical compositions comprising an effective amount of an anti-TNFα antibody, or antigen-binding portion thereof, and a pharmaceutically acceptable carrier, wherein the effective amount of the anti-TNFα antibody may be effective to treat a TNFα-related disorder, including, for example, Crohn's disease. In one embodiment, the antibody or antibody portion is incorporated into a pharmaceutical formulation as described in PCT/IB03/04502 and U.S. application Ser. No. 10/222,140, incorporated by reference herein. This formulation includes a concentration 50 mg/ml of the antibody adalimumab, wherein one pre-filled syringe contains 40 mg of antibody for subcutaneous injection.

The antibodies, or antibody-portions, obtained using the methods of the present invention can be administered by a variety of methods known in the art, although for many therapeutic applications, the preferred route/mode of administration is subcutaneous injection. In another embodiment, administration is via intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

The antibodies, or antigen-binding portion thereof, obtained using the methods of the invention can also be administered in the form of protein crystal formulations which include a combination of protein crystals encapsulated within a polymeric carrier to form coated particles. The coated particles of the protein crystal formulation may have a spherical morphology and be microspheres of up to 500 micro meters in diameter or they may have some other morphology and be microparticulates. The enhanced concentration of protein crystals allows the antibody of the invention to be delivered subcutaneously. In one embodiment, the antibodies of the invention are delivered via a protein delivery system, wherein one or more of a protein crystal formulation or composition, is administered to a subject with a TNF-related disorder. Compositions and methods of preparing stabilized formulations of whole antibody crystals or antibody fragment crystals are also described in WO 02/072636, which is incorporated by reference herein. In one embodiment, a formulation comprising the crystallized antibody fragments described in PCT/IB03/04502 and U.S. application Ser. No. 10/222,140, incorporated by reference herein, are used to treat a TNFα-related disorder using the multiple-variable dose methods of the invention. In certain embodiments, an antibodies, or antigen-binding portion thereof, obtained using the methods of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or coadminister the compound with, a material to prevent its inactivation. The pharmaceutical compositions of the invention may include a “therapeutically effective amount” or a “prophylactically effective amount” of an antibody or antigen-binding portion thereof of the invention. A “therapeutically effective amount” refers to an amount effective, at dosages and for periods of time necessary, to achieve the desired therapeutic result. A therapeutically effective amount of the antibody, or antigen-binding portion thereof, may vary according to factors such as the disease state, age, sex, and weight of the individual, and the ability of the antibody, antibody portion, other.

A therapeutically effective amount is also one in which any toxic or detrimental effects of the antibody, or antigen-binding portion thereof, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount. Dosage regimens may be adjusted to provide the optimum desired response {e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit comprising a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of an antibody, or antigen-binding portion thereof, is 10 to 200 mg, more preferably 20 to 160 mg, more preferably 40 to 80 mg, and most preferably 80 mg. In one embodiment, the therapeutically effective amount of an antibody or, antigen-binding portion thereof, is about 20 mg. In another embodiment, the therapeutically effective amount of an antibody or portion thereof is about 40 mg. In still another embodiment, the therapeutically effective amount of an antibody or, antigen-binding portion thereof, is about 80 mg. In one embodiment, the therapeutically effective amount of an antibody or portion thereof for use in the methods of the invention is about 120 mg. In yet another embodiment, the therapeutically effective amount of an antibody, or antigen-binding portion thereof, is about 160 mg. Ranges intermediate to the above recited dosages, e.g. about 78.5 to about 81.5; about 15 to about 25; about 30 to about 50; about 60 to about 100; about 90 to about 150; about 120 to about 200, are also intended to be part of this invention. For example, ranges of values using a combination of any of the above recited values as upper and/or lower limits are intended to be included.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Antibodies, or antibody-portions thereof, obtained using the methods of the invention may be administered on a biweekly dosing regimen as described in WO02/100330, a low dose regimen as described in WO 04/037205, and a multiple variable dosing regimen as described in WO 05/110452, each of which is incorporated by reference herein.

The invention also pertains to packaged pharmaceutical compositions, articles of manufacture, or kits comprising the antibody, or antigen-binding portion thereof, obtained using the process of the invention. The article of manufacture may comprise an antibody, or antigen-binging portion thereof, obtained using the method of the invention and packaging material. The article of manufacture may also comprise label or package insert indicating the formulation or composition comprising the antibody, or antigen-binding portion thereof, has a reduced level of HCP and/or procathepsin L. The article of manufacture may comprise a label or package insert contained within the packaging material indicating that the adalimumab formulation comprises no greater than about 70 ng/mg of HCP or a label or package insert contained within the packaging material indicating that the adalimumab formulation comprises no greater than about 13 ng/mg. The article of manufacture may comprise a label or package insert contained within the packaging material indicating that the adalimumab formulation comprises no greater than about 5 ng HCP/mg adalimumab. The article of manufacture may also comprise packaging material indicating that the adalimumab formulation comprises no greater a level of procathepsin L than that indicated by a cathepsin L activity of about 3.0 RFU/s/mg adalimumab.

The antibodies purified using the methods described herein can be used for inhibiting TNFα activity in a subject suffering from a disorder in which TNFα activity is detrimental. TNFα has been implicated in the pathophysiology of a wide variety of disorders (see e.g., Moeller, A., et al. (1990) Cytokine 2:162-169; U.S. Pat. No. 5,231,024 to Moeller et al; European Patent Publication No. 260610 B1 by Moeller, A.) including sepsis, infections, malignancies, autoimmune diseases, pulmonary disorders, intestinal disorders, transplant rejection and graft-versus-host disease.

Examples of autoimmune conditions include rheumatoid arthritis, rheumatoid spondylitis, osteoarthritis and gouty arthritis, allergy, multiple sclerosis, autoimmune diabetes, arthritis, metabolic diseases, autoimmune uveitis, lupus, Crohn's disease, cardiac disorders and nephrotic syndrome. Other examples of autoimmune conditions include multisystem autoimmune diseases and autoimmune hearing loss. A more extensive list of diseases which may be treated using the antibodies purified according to the present methods is set forth in WO2007117490, the contents of which is incorporated herein.

It is expected that during the life of a patent maturing from this application many relevant anti-TNFalpha antibodies will be developed and the scope of the term anti-TNFalpha antibody is intended to include all such new technologies a priori.

As used herein, the terms “about” or “approximately” used with a pH or pI (isoelectric point) value refers to a variance of 0.1, 0.2, 0.3, 0.4 or 0.5 units. When used with a temperature value, “about” or “approximately” refers to a variance of 1, 2, 3, 4 or 5 degrees. When used with other values, such as length and weight, “about” or “approximately” refers to a variance of 1%, 2%, 3%, 4% or 5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### MATERIALS

Table 1, herein below summarizes the “in process” control samples that have to be collected during the purification.

TABLE 1

| IPC/IPA samples to be | Sample | Purification | Steps |
|---|---|---|---|
| Mab load | 1.0 | MabSelect Sure | 1 |
| Mab unbound | 1.1 | | |
| Mab wash | 1.2 | | |
| Mab elution | 1.3 | | |
| V.I. | 2.0 | V.I. | 2 |
| Eshmuno S unbound | 3.0 | Eshmuno S | 3 |
| Eshmuno S (HEPES wash) | 3.1 | | |
| Eshmuno S F1 | 3.2 | | |
| Eshmuno S Elution | 3.3 | | |
| Eshmuno S F3 | 3.4 | | |
| Capto Adhere load | 4.0 | Capto Adhere | 4 |
| Capto Adhere unbound | 4.1 | | |
| UF filtrate A (of the first concentration) | 5.0 | UF, 50 kDa | 5 |
| UF filtrate B (of the dialysis stage) | 5.1 | | |
| UF retentate | 5.2 | | |
| U18 API | 6.0 | 0.2 filtration | 6 |

### Example 1

### Method for Purifying Adalimumab

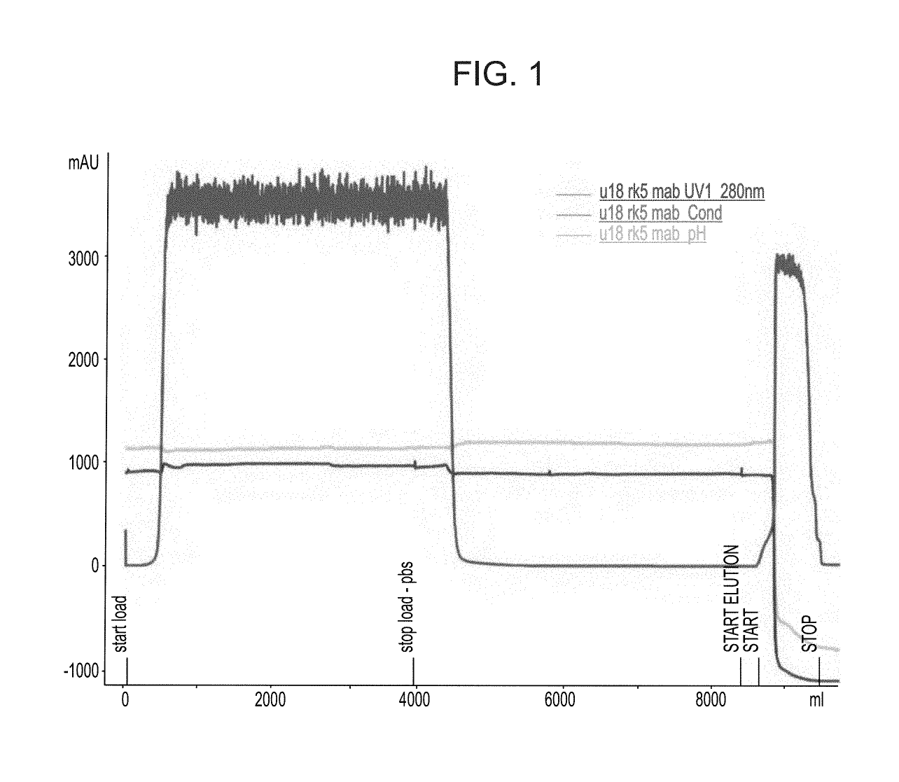

A flow diagram depicting an exemplary method for purifying Adalimumab is presented in FIG. 1.

Crude Harvest:

1 L of “Clarified Adalimumab crude harvest” is used.

Purification of Adalimumab by Affinity Chromatography on a MabSelect SuRe Column

Column Set-Up:

Attach the GE Healthcare column packed with 490 ml (5 cm I.D.×25 cm L) MabSelect Sure (GE Healthcare) resin to the AKTA™ Pilot (GE Healthcare).

Set the maximal pressure at 0.3 MPa.

Wash the column (removing the 20% Ethanol) in “down flow” direction with 3 CV of water at flow rate of 164 ml/minute (500 cm/h).

Column Run:

Wash the column with 5 CV of 20 mM phosphate buffer, 150 mM NaCl, pH 7.4 at a flow rate of 164 ml/minute.

Load the “Mab load #1.0”, containing a maximum of 19.6 g Adalimumab (40 mg/mL resin) at maximum flow rate of 82 ml/minute (250 cm/hour) at R.T.

Minimum residence time should be NLT 6 minutes.

Take a 5 ml sample from the load for IPC analysis. Label the sample “Mab load #1.0” (see Table 1).

Wash the column with 20 mM phosphate buffer, 150 mM NaCl, pH 7.4, at a flow rate of 82 ml/minute. When the absorbance at 280 nm returns to a low and stable reading, take a 5 mL sample for IPC reading from the Unbound.

Continue to wash the column with 20 mM phosphate buffer, 150 mM NaCl, pH 7.4, at a flow rate of 164 ml/minute (500 cm/hour) up to 10 CV from the beginning of the wash step (3.2.2.4).

Collect the Wash sample for analysis.

Take a sample for analysis, label the sample: “Wash #1.2”.

Elute Adalimumab with 100 mM acetate buffer pH 3.6, at a flow rate of 82 ml/minute (250 cm/hour).

Collect the Adalimumab peak into a 5 L bottle labeled “Ab elution #1.3”. Start collecting when the absorbance at 280 nm reaches 10 mAU OD 280 nm.

Stop collecting when absorbance value returns to 40 mAU OD 280 nm.

Take a sample from the collected elution for IPC. Label the sample: “Mab elution #1.3”.

Perform visual inspection of the elution fraction (#1.3).

Next step (V.I.) will be done at the same day as the affinity chromatography.

Column Regeneration:

Perform the regeneration at “up flow” mode.

Wash the column with 3 CV of 0.5 M acetic acid, pH 3.0 at a flow rate of 164 ml/minute (500 cm/hour).

Wash the column with 3 CV of 20 mM phosphate buffer, 150 mM NaCl, pH 7.4, at a flow rate of 164 ml/minute (500 cm/hour).

Wash the column with 5 CV of 0.1 M NaOH, at a flow rate of 164 ml/minute (500 cm/hour).

Wash the column with 20 mM phosphate buffer, 150 mM NaCl, pH 7.4 until a stable pH reading of pH 7.4 is reached, at a flow rate of 164 ml/minute (500 cm/hour).

Wash the column with water until conductivity decreases below <0.1 mS/cm², at a flow rate of 164 ml/minute (500 cm/hour).

Column is stored in 20% Ethanol at 2-8° C.

Viral Inactivation Step

The procedure is performed at R.T.

Lowering the pH Levels:

Start lowering pH level of Mab elution #1.3 slowly to pH 3.6 with 0.5 M Acetic acid.

Store the bottle at room temperature (21-25° C.) for 60 minutes.

Neutralizing the pH to pH 4.5:

Raise the pH level slowly to pH 4.6 with 0.5 M Sodium Acetate.

Dilute the sample with water to conductivity 4.75 mS/sec with water

Change the label of the 5 L bottle to “V.I. #2.0”.

Store at 2-8° C., until the next day.

Purification of Adalimumab by Cation Exchanger Chromatography on Eshmuno™ S Column

Sample Preparation:

Filter the solution through Durapore PVDF 45 um.

### Column Set-Up:

Attach column (Millipore) packed with 304 ml (4.4 cm I.D.×20 cm L) Eshmuno® S resin (Merck) to the AKTA—Pilot.

Set the maximal pressure at 0.7 Mpa (resin is stable up to 0.8 Mpa).

Perform the purification step in a “down-flow” mode.

Wash the column with 3 CV of water at a flow rate of 127 ml/minute (500 cm/h).

Wash the column with 5 CV of 20 mM citrate buffer, 1 M sodium chloride pH 5.2 at a flow rate of 127 ml/minute (500 cm/h), until the conductivity and pH are the same as those of the buffer.

Prime AKTA “B” pump with 20 mM citrate buffer, 0.4 M sodium chloride pH 5.2.

Column Run:

Equilibrate the column with 5 CV of 20 mM citrate buffer pH 5.2 at a flow rate of 127 ml/minute (500 cm/h).

Check that the absorbance reading (at 254 nm), pH and the system pressure are stabilized at a flow rate of 40 ml/minute (150 cm/hour).

Perform auto zero to the absorbance.

Load Adalimumab solution containing 6-11.5 g Adalimumab from the bottle labeled “V.I #2.0” on the Eshmuno S column, at a flow rate of 40 ml/minute (150 cm/hour).

Collect the unbound fraction in a 5 L bottle labeled “Eshmuno S unbound #3.0).

Wash the column with 3 CV of 20 mM Citrate buffer pH 5.2 at a flow rate of 40 ml/minute (150 cm/hour).

Wash the column with 2 CV of 10 mM HEPES pH 6.8 at a flow rate of 127 ml/minute (500 cm/h).

Wash the column with 10 CV of 10 mM HEPES pH 8.0 at a flow rate of 127 ml/minute (500 cm/h).

Wash the column with 4 CV of 10 mM HEPES pH 6.8 at a flow rate of 127 ml/minute (500 cm/h).

Wash the column with 3 CV of 15% Buffer B at a flow rate 127 ml/minute (500 cm/h).

Perform auto zero to the absorbance.

Wash the column with 1 CV of 15% Buffer B at a flow rate of 40 ml/minute (150 cm/hour).

Elute the Adalimumab with the gradient program as set forth in Table 2.

TABLE 2

| (%) “B” | (%) “A” | Flow rate ml/min | CV |
|---|---|---|---|
| 15 | 85 | 40 | Initial |
| 100 | 0 | 40 | 17 |

A—20 mM citrate buffer pH 5.2.

B—20 mM vitrate buffer pH 5.2, 0.4 M NaCl

Collect “pre-elution” fraction (F1) to bottle labeled “Eshmuno S F1 #3.1” when the absorbance at 254 nm starts to rise.

Start collecting the Adalimumab peak (when the absorbance at 254 nm reaches 200 mAU), into 5.0 L bottle labeled “Eshmuno S elution #3.2”.

Stop collecting the peak when, on the down slope, the absorbance at 254 nm reaches 30% from the peak maximum.

Calculate the fraction weight by weighing the 5.0 L bottle containing the fraction and subtract the bottle weight (Tara).

Take a sample from the Eshmuno S elution fraction for IPC. Label the sample: “Eshmuno S elution #3.2”.

Column Regeneration:

Perform the regeneration at “up flow” mode.

Wash the column with 3 CV of 20 mM citrate buffer pH 5.2, 1 M NaCl, at a flow rate of 127 ml/minute (500 cm/h).

Wash the column with 4 CV of 1 M NaOH at a flow rate of 127 ml/minute (500 cm/h).

Wash the column with 20 mM citrate buffer pH 5.2, 1 M NaCl, at a flow rate of 127 ml/minute (500 cm/h), until stable pH reading is reached.

Wash the column with water until conductivity decreases below <0.1 mS/cm², at a flow rate of 127 ml/minute (500 cm/h).

Column is stored in 20% Ethanol at room temperature.

Purification of Adalimumab by Mixed-Mode Chromatography on a Capto Adhere Column

Sample Preparation:

Adalimumab load should be 100 mg/ml Capto Adhere (GE Healthcare) resin.

Therefore, on the small scale purification (GE Healthcare XK 26/40 column packed with ˜100 ml (2.6 cm I.D×19 cm L) Capto Adhere resin) 10 g should be loaded.

If necessary, adjust the temperature of “Eshmuno S elution #3.2” solution to room temperature (22-25° C.).

Gently, stir the solution with a magnetic bar on a magnetic stirrer.

Titrate the solution, avoiding the formation of foam with 500 mM phosphate dibasic to “Eshmuno S elution #3.2” to pH 7.8˜0.5/1 v/v.

Correct the conductivity of the solution to 17 mS/cm², with water ˜0.5/1 v/original volume.

Change the label of the “Eshmuno S elution #3.2″bottle to “Capto Adhere load #4.0”.

Take a sample from the Capto Adhere load fraction for IPC. Label the sample: “Capto Adhere load #4.0”.

Column Set-Up:

Attach the GE Healthcare XK 26/40 column packed with ˜100 ml (2.6 cm I.D×19 cm L) Capto Adhere (GE Healthcare) resin to the AKTA Pilot.

Set the maximal pressure at 0.3 MPa.

Perform the purification step in a “down-flow direction” mode.

Wash the column with 3 CV of water at a flow rate of 20 ml/minute (230 cm/h).

Wash the column with ˜5 CV of 50 mM phosphate buffer, 1 M sodium chloride, pH 7.8 at a flow rate of 20 ml/minute, until the conductivity and pH are the same as those of the buffer.

Column Run:

Equilibrate the column with 5 CV of 50 mM phosphate buffer, pH 7.8 at a flow rate of 20 ml/minute (230 cm/h).

Check that the absorbance reading, pH and the system pressure are stabilized.

Load “Capto Adhere load #4.0” solution containing ˜10 g on the Capto Adhere column (100 mg Adalimumab/ml resin) at a flow rate of 20 ml/minute (230 cm/h).

Start collecting the unbound fraction containing Adalimumab when absorbance at 280 nm reaches ˜10 mAU, into a 10 L bottle labeled “Capto-Adhere unbound #4.1”.

Stop collecting the peak when, on the down slope, peak absorbance reaches ˜200 mAU.

Take a sample from the Capto Adhere unbound fraction for IPC. Label the sample: “Capto Adhere unbound #4.1”.

Titrate the solution with 1M citric acid to pH 4.5.

Take a sample from the Capto Adhere unbound fraction for IPC. Label the sample: “UF load #4.2”.

Keep the solution cold at 2-8° C.

Column Regeneration

Perform regeneration process in an “up-flow direction” mode.

Wash the column with 0.1 M acetate pH 3.0, at a flow rate of 20 ml/minute, until the peak elutes completely.

Wash the column with additional 2 CV of 0.1 M acetate pH 3.0, at a flow rate of 20 ml/minute.

Wash the column with 3 CV of 50 mM phosphate buffer, 1M sodium chloride, pH 7.8, at a flow rate of 20 ml/minute.

Wash the column with 1 M NaOH, at a flow rate of 20 ml/minute. Set contact time for 15-30 minutes (0.3-0.6 L 1 M NaOH).

Wash the column with 50 mM phosphate buffer, 1 M sodium chloride, pH 7.8, at a flow rate of 20 ml/minute, until stable pH reading is reached.

Wash the column with water until conductivity decrease below <0.1 mS/cm², at a flow rate of 20 ml/minute.

Column is stored in 20% Ethanol in room temperature.

UF Adalimumab

For this step, use three 50 kDa ultra-filtration Kvick Lab Packet, 0.01 m² membranes (GE). The concentration (to 25 mg/ml), DF and is performed by the AKTA crossflow in TPM=1.0 Bar. The “final concentration” is performed by the AKTA crossflow at TPM=0.2 Bar, circulate for 20 min after reaching the desired concentration. The retentate flux 360 LMH (retentate flow of 180 ml/min using 3×0.01 m² membranes).

Membrane Regeneration:

The regeneration is performed by AKTAcrossflow.

Regeneration steps include;

1) Wash the membrane with 1 M NaCl

2) Circulate for 1 Hr.

3) Wash the membrane with 300 ppm Na hypochlorite in NaOH 0.5 M

4) Circulate for 1 Hr.

5) Wash the membrane water ˜850 ml.

6) Wash the membrane with 0.2% phosphoric acid at pH 4

7) Circulate for 1 Hr.

8) Wash the membrane with water ˜850 ml.

9) Perform NWP.

10) Store the filter at 0.1M NaOH.

Filtration of Adalimumab using a 0.2 μm membrane (should be performed on the same day as the UF)

Filter the solution through a Stericup (Millipore)—PES 0.22 μm filter into a 0.5 L-bottle labeled “Adalimumab API”.

Measure the absorbance of “Adalimumab API” at 280 nm and determine the total amount (mg) according to the following equation:

E₂₈₀ nm 1.39 (mg/ml)⁻¹ cm⁻¹=1 mg/ml

Total Adalimumab amount (mg)=OD at 280 nm (cm⁻¹)/1.39×total volume (ml).

Keep the “Adalimumab API” solution at 2-8° C.

Results

The purification of adalimumab by Affinity Chromatography on a MabSelect SuRe Column is illustrated in FIG. 1.

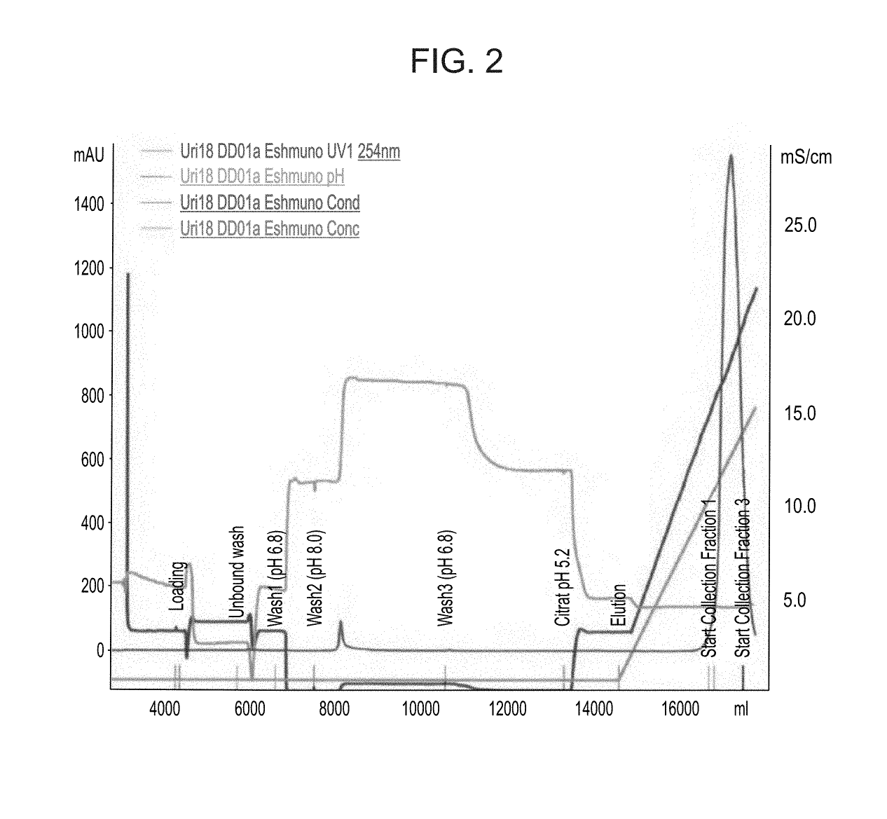

The purification of adalimumab by Cation-Exchange Chromatography on an Eshmuno-S column is illustrated in FIG. 2.

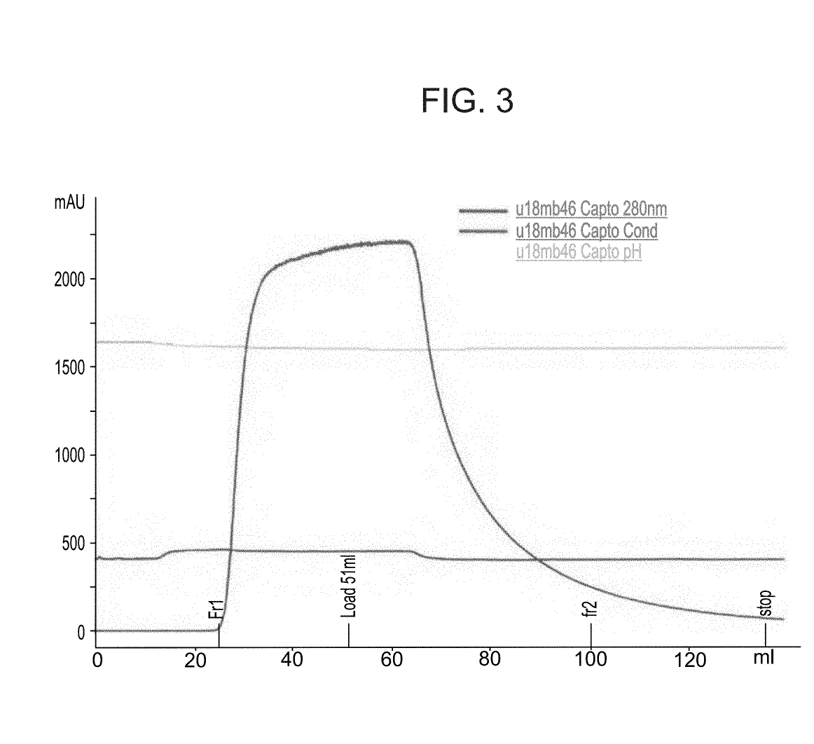

The purification of adalimumab by Mixed mode Chromatography on a Capto Adhere Column, Flow-Through Mode is illustrated in FIG. 3.

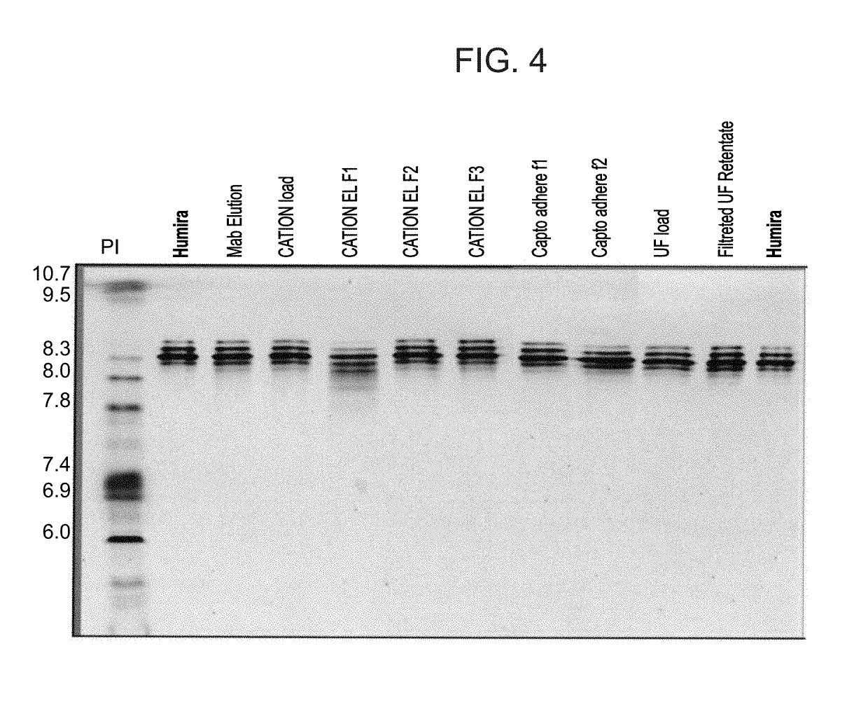

Isoelectric Focusing of samples from the purification process of adalimumab is illustrated in FIG. 4.

Mass Balance of adalimumab is summarized in Table 3, herein below.

TABLE 3

| Step recovery (%) | Purification step |
|---|---|
| 89-98 | Mab |
| 84-92 | Eshmuno-S |
| 84-96 | Capto |
| 85-92 | 50 kDa Retentate |

Purity of adalimumab is summarized in Table 4, herein below.

TABLE 4

| HCP (ppm) | Aggregates (%) | Purification step |
|---|---|---|
| 514-1124 | 2.32-2.84 | Mab |
| 36-82 | 0.83-1.1 | Eshmuno-S |
| ≦1.5 | 0.07-0.16 | Capto |
| ≦0.82 | 0.17-0.38 | 50 kDa Retentate |

Appearance of the solution at the end of every purification step as determined by visual inspection is described in the Table 5 herein below.

TABLE 5

| Visual inspection | Purification step |
|---|---|
| Clear with slight yellow color | mAbSelect SuRe, Elution |
| Clear with slight yellow color | Eshmuno-S |
| Clear, colorless | Capto Adhere, Unbound |
| Opalescent, colorless | 50 kDa Ultrafiltration, Retentate |
| Clear, colorless | API 0.22 μm Filtration |

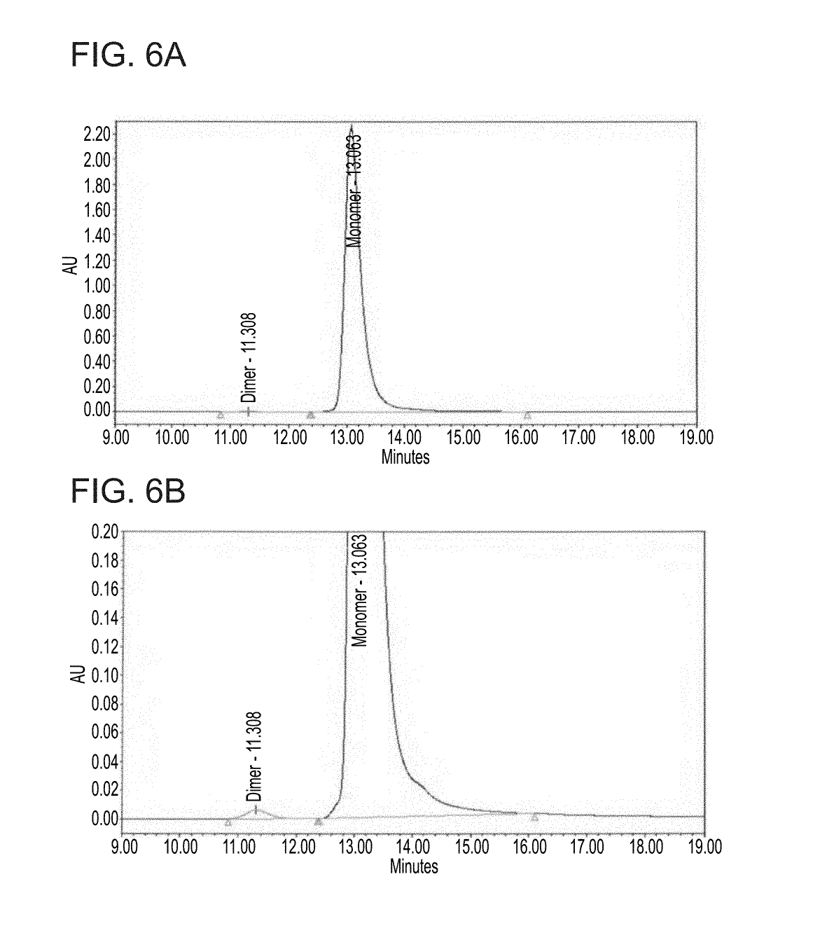

Purity estimation by size exclusion HPLC of adalimumab, is illustrated in FIGS. 6A-B.

The area (%) of each peak for adalimumab is summarized in Table 6 herein below.

TABLE 6

| Monomer | Dimer | |
|---|---|---|
| 99.2-99.6 | 0.2-0.4 | Humira range |
| 99.6 | 0.4 | InSight-Adalimumab |

The Humira range is based on seven Humira lots: #13252XH03, #25365XH07, 13259XD13, 15278XH04, 28390XH02, 28390XH04 and 28390XH08.

Quantitation of CHO Host Cell Proteins Present in Adalimumab by ELISA:

Host Cell Proteins level was determined using the 3^{rd} Generation ELISA kit for CHO HCP detection (Cygnus Technologies). Adalimumab sample diluted ×2 with diluent buffer and CHO HCPs standards was reacted simultaneously with a horseradish peroxidase (HRP) enzyme labeled anti-CHO antibody (goat polyclonal) in microtiter strips coated with an affinity purified capture anti-CHO antibody. The immunological reactions result in the formation of a sandwich complex of solid phase antibody-HCP-enzyme labeled antibody. The microtiter strips are washed to remove any unbound reactants. The substrate, tetramethyl benzidine (TMB) is then reacted. The amount of hydrolyzed substrate is read on a microtiter plate reader at 450 nm and is directly proportional to the concentration of CHO HCPs present.

Adalimumab contains an HCP level of NMT ˜1 parts per million (ppm).

### Example 2

### Method for Purifying Infliximab

Purification Process of InSight-Infliximab

Approximately 2.8 g Infliximab were loaded onto a 64 ml affinity column of MabSelect SuRe (≦45 mg/ml resin, GE Healthcare), where the antibody is captured and partially purified. Prior to the loading the column was equilibrated with 5 CV of 20 mM Phosphate buffer, 150 mM NaCl, pH 7.4. The load and equilibration flow rate was 63 cm/hour or less. The residence time was NLT 10 minutes.

The column was washed with 20 mM Sodium Phosphate buffer, 1 M NaCl, 0.05% Triton, pH 7.4 and then, with 100 mM Acetate buffer pH 5.0. The antibody was eluted with 100 mM Acetate buffer pH 3.6. The pH of the eluted fraction was ˜4.0-4.2. The washes and the elution were done at a flow rate of 63 cm/hour.

The column underwent regeneration by washing with 3 CV of 0.5 M Acetic acid, pH 3.0 followed by 3 CV of 20 mM Phosphate buffer, 150 mM NaCl, pH 7.4, exposure for 20 minute to 0.1 M NaOH or 50 mM NaOH, 0.5 M Na2SO4 and neutralization of the pH with 20 mM Phosphate buffer, 150 mM NaCl, pH 7.4.

Eluted fraction of MabSelect SuRe column was then acidified to pH 3.8 and incubated for 30 minutes at room-temperature (22-25° C.) for viral inactivation (VI). The pH of Infliximab was adjusted to pH 5.8 with 0.5 M Sodium Acetate solution.

2.2 g of the InSight-Infliximab intermediate was loaded on a 44 ml Cation Exchange column of Eshmuno-S (≦50 mg/ml resin, Merck) at a flow rate of 150 cm/hour. Prior to the load the column was washed with water followed by 5 CV of 5 mM Phosphate buffer, 1 M Sodium Chloride pH 7.2 at room temperature and then equilibrated with 10 CV of 20 mM Acetate buffer pH 5.8.

Following loading, the column was washed with 3 CV of 20 mM Acetate buffer pH 5.8, 0-12 CV of 5 mM Phosphate buffer pH 7.2 and by 3 or 12 CV of 10 mM Phosphate buffer pH 6.0. The antibody was then eluted with a gradient of 20 or 40 CV from 0 to 100% of 0.3 M Sodium Chloride in 10 mM Phosphate buffer pH 6.0.

The antibody solution was adjusted to a concentration of 14 to 7 or precisely 2 mg/ml in 50 mM Phosphate buffer, pH 7.0 and 1.76 g or 1.32 g InSight-Infliximab were loaded on 22 ml mixed-mode Capto Adhere column (80 or 60 mg/ml resin respectively, GE Healthcare). The column was washed with 50 mM Phosphate buffer, pH 7.0 and with 50 mM Phosphate buffer, pH 6.1. The unbound and eluted fractions were pooled together.

The antibody solution from the Capto Adhere chromatography step was concentrated and dialyzed against 10 mM monobasic Phosphate, using a KVICKLAB 1.2FT2, 50KD, PES, 0.02 m² membrane (GE health care).

Column geometry and flow rates are summarized in Table 7, herein below.

TABLE 7

| Production scale | | | pilot scale | | | R&D scale | | | |
|---|---|---|---|---|---|---|---|---|---|
| Capto | Eshmuno | Mab | Capto | Eshmuno | Mab | Capto | Eshmuno | Mab | |
| 12 | 18 | 12.7 | 11 | 22 | 105 | 11 | 22 | 12 | Height |
| | | | | | | | | | (cm) |
| 44.6 | 44.6 | 60 | 10 | 10 | 14 | 1.6 | 1.6 | 2.6 | Diameter |
| | | | | | | | | | (cm) |
| 1562 | 1561 | 2827 | 79 | 79 | 154 | 2 | 2 | 5.3 | Cross section |
| | | | | | | | | | (cm²) |
| 18747 | 28107 | 35908 | 880 | 1688 | 1600 | 22 | 44 | 63 | Volume |
| | | | | | | | | | (cm³) |
| 3.4 | 9 | 11 | 3.8 | 9 | 11 | 2.9 | 8.8 | 10 | Resident time, |
| | | | | | | | | | (min) |
| 230 | 120 | 72 | 230 | 150 | 63 | 230 | 150 | 63 | Flow rate |
| | | | | | | | | | (cm/hr) |
| 5983 | 3123 | 3396 | 300 | 195 | 160 | 7.7 | 5 | 5.6 | Flow rate |
| | | | | | | | | | (cm³/min) |

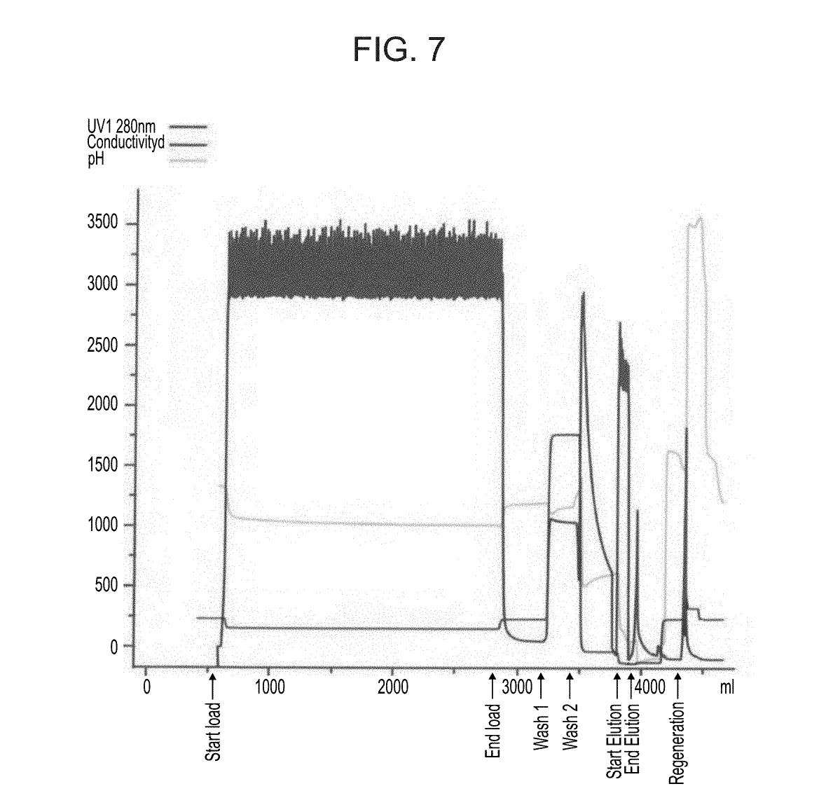

Purification of Infliximab by Affinity Chromatography on a MabSelect SuRe Column is illustrated in FIG. 7.

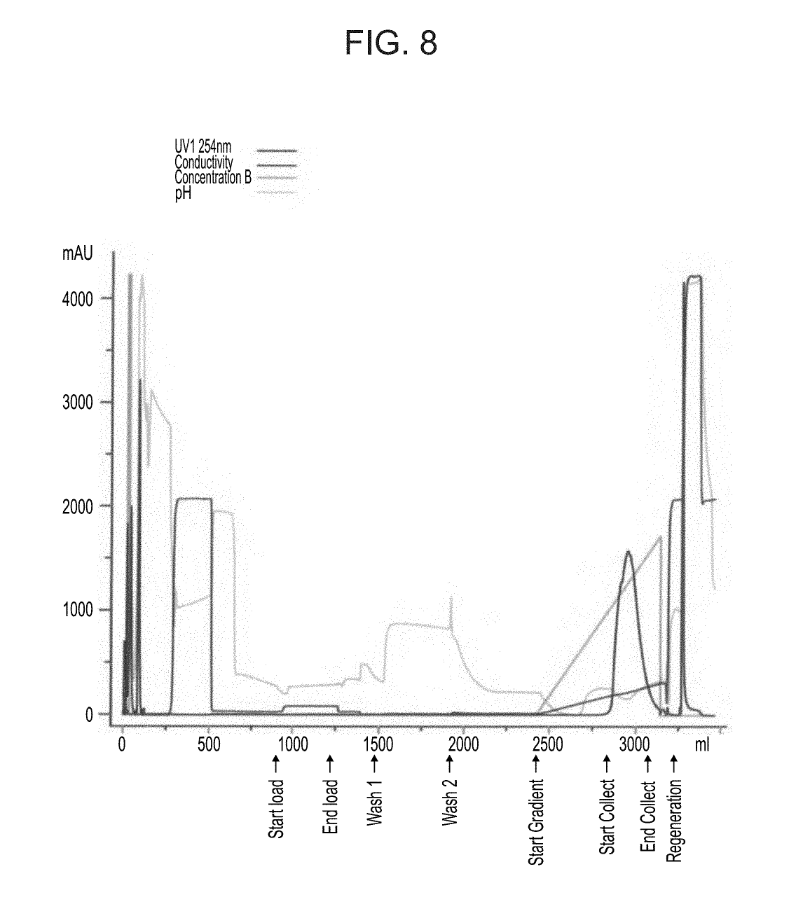

Purification of Infliximab by Cation-Exchange Chromatography on an Eshmuno-S column is illustrated in FIG. 8.

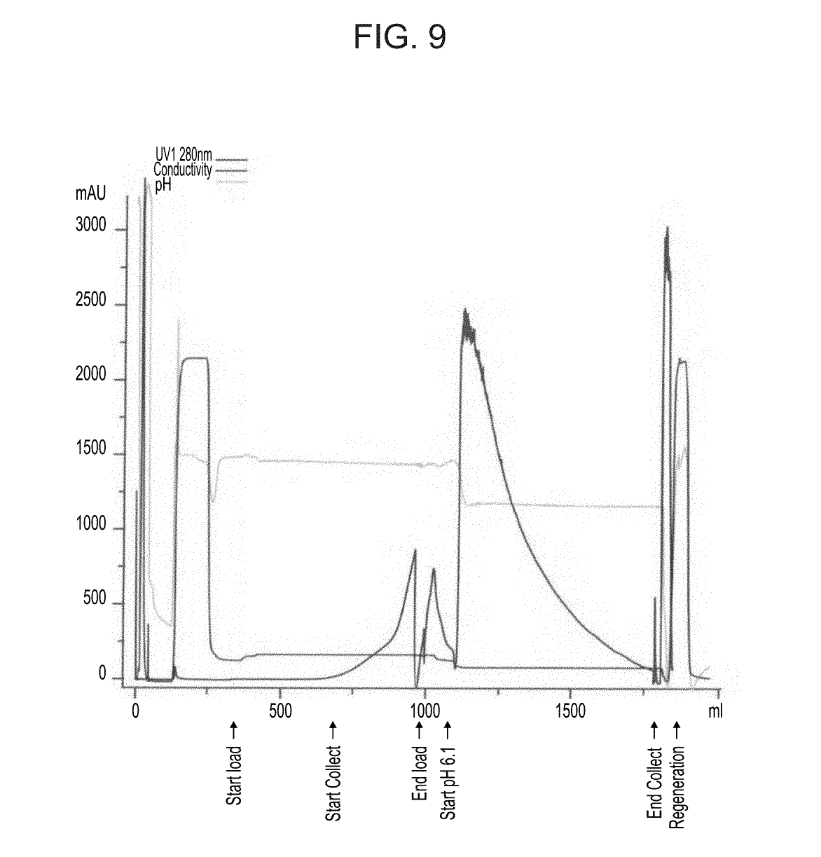

Purification of Infliximab by Mixed mode Chromatography on a Capto Adhere Column, Flow-Through Mode is illustrated in FIG. 9.

A visual inspection of the solution at the end of every purification step was performed and the observations are described in the Table 8 below.

TABLE 8

| Visual inspection | Purification step |
|---|---|
| Clear with slight yellow | MabSelect SuRe, |
| color | Elution |
| Opalescent with slight | Eshmuno-S |
| yellow color | |
| Clear, colorless | Capto Adhere, Unbound |
| Opalescent, colorless | 2^{nd} 50 kDa |
| | Ultrafiltration, Retentate |
| Clear, colorless | API 0.22 μm Filtration |

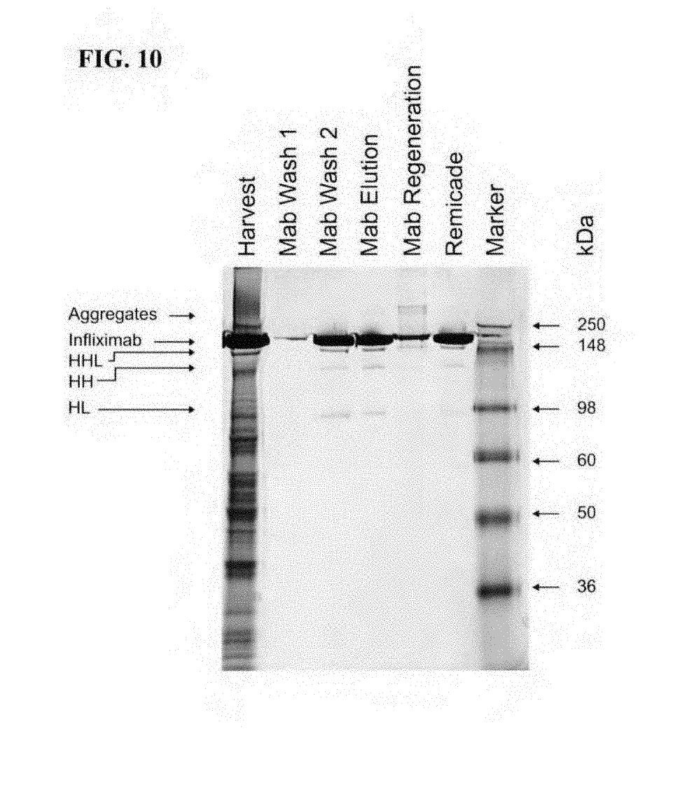

In-Process Control SDS-PAGE Analysis of InSight-Infliximab Samples from the MabSelect SuRe Column is illustrated in FIG. 10.

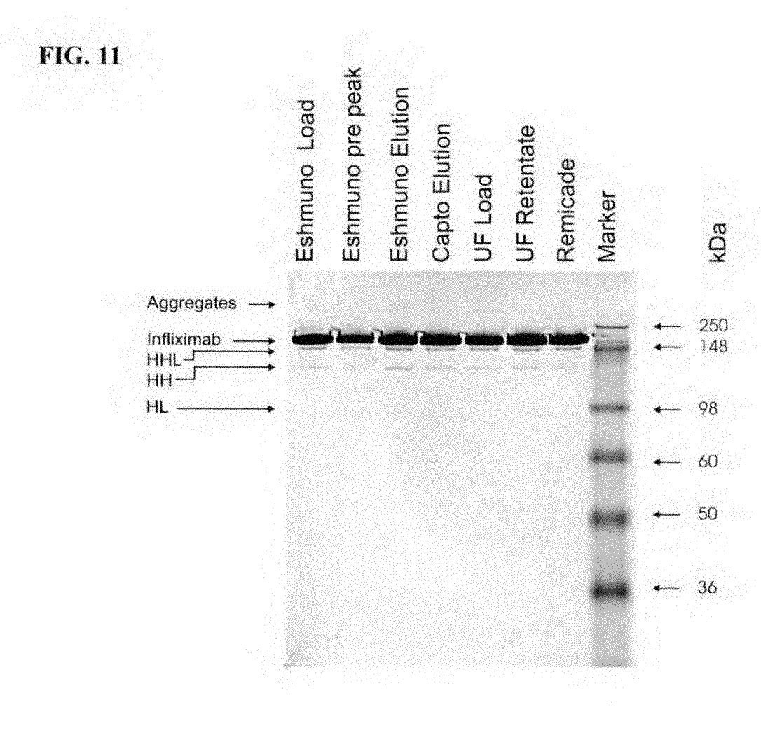

In-Process SDS-PAGE Analysis of InSight-Infliximab Samples from Cation-Exchange Chromatography on an Eshmuno-S Column to Ultrafiltration is illustrated in FIG. 11.

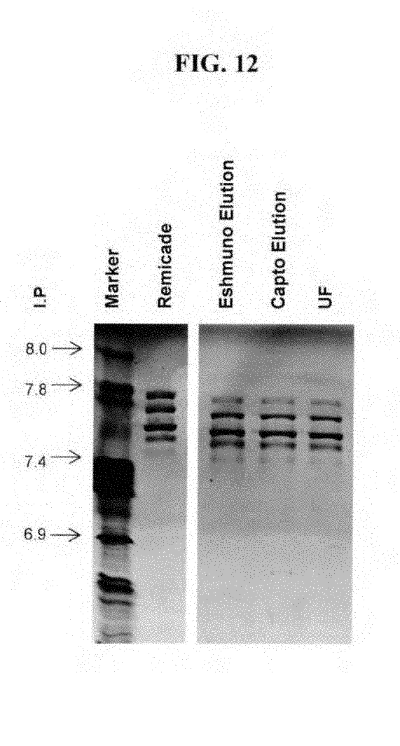

Isoelectric focusing for Infliximab and Remicade is illustrated in FIG. 12.

Mass Balance of Infliximab is summarized in Table 9.

TABLE 9

| Step recovery (%) | Purification step |
|---|---|
| 77-95% | Mab |
| 77-94 | Eshmuno-S |
| 66-91 | Capto |
| 78-92 | 50 kDa Retentate |

Purity of Infliximab is summarized in Table 10.

TABLE 10

| HCP (ppm) | Aggregates (%) | Purification step |
|---|---|---|
| 5989-1432 | 2.59-7.47 | Mab |
| 568-58 | 1.74-0.9 | Eshmuno-S |
| ≦2.9 | 0.39-0.15 | Capto |
| ≦1.8 | 0.48-0.18 | 50 kDa Retentate |

Table 11 below summarizes the ranges of four pilots and four production scale runs.

| Ranges | Purification step |
|---|---|
| | MabSelect Sure |
| 22-51 | Load mg/ml resin |
| 18-25 | Start load temperature (° C.) |
| 4.01-4.15 | Elution pH |
| 6.0-24.5 | Elution concentration (mg/ml) |
| 1.4-3.5 | Elution volume (CV) |
| | V.I |
| 3.0-3.8 | V.I. volume (times Mab elution) |
| | 1^{st} UF (alternatively) |
| <240 | Load g/m² membrane |
| 10-25 | Start filtration temperature (° C.) |
| Same as load | UF retentate concentration |
| (V.I) | |
| | Eshmuno-S* |
| 32-50 | Load mg/ml resin |
| 15.5-25 | Start load temperature (° C.) |
| 1.51-2.6 | Start collect conductivity |
| 2.4-5.8 | End collect conductivity |
| 2.2-3.7 | Elution concentration |
| 8.8-14.3 | Elution volume (CV) |
| | Capto Adhere |
| 21.5-31.0 | Load volume (CV) |
| 43-62 | Load mg/ml resin |
| 21.3-25 | Start load temperature (° C.) |
| 0.7-1.7 | Elution concentration |
| | Elution volume |
| | UF |
| 81-100 | Load g/m2 membrane |
| 11.8-25 | Start filtration temperature (° C.) |
| 33.4-58.3 | UF retentate concentration |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of purifying an antibody (Ab) from a mixture comprising impurities comprising:
(a) purifying the Ab using an affinity resin;
(b) purifying the Ab using a cation exchange (CEX) resin; and
(c) purifying the Ab using a mixed mode (MM) resin which comprises anion exchange and hydrophobic interaction functional groups; wherein step (b) follows step (a) and step (c) follows step (b), and wherein the method does not comprise use of a hydrophobic interaction chromatography (HIC) medium, thereby purifying the Ab.

2. A method of purifying an antibody (Ab) from a mixture comprising impurities comprising:
(a) purifying the Ab using an affinity resin;
(b) purifying the Ab using a mixed mode (MM) resin which comprises anion exchange and hydrophobic interaction functional groups;
(c) purifying the Ab using a CEX membrane adsorber, wherein the method does not comprise use of a hydrophobic interaction chromatography (HIC) medium.

3. The method of claim 1, wherein said affinity resin comprises a protein A resin or a protein A resin comprising mABSelect SuRe™.

4. The method of claim 1, not comprising contacting the Ab with an anion exchange (AEX) chromatography medium.

5-7. (canceled)

8. The method of claim 1, wherein the Ab is an antibody to tumor necrosis factor (TNF) selected from the group consisting of adalimumab, infliximab and a biosimilar to adalimumab or infliximab.

9. (canceled)

10. The method of claim 1, further comprising performing a viral inactivation step following step (a) and prior to step (b).

11. The method of claim 10, wherein said viral inactivation step is effected by lowering the pH of said first eluate to a pH between 3 and 4.

12. (canceled)

13. The method of claim 1, wherein said CEX resin comprises a SO₃ functional group or said CEX resin comprises a SO₃ functional group comprising Eshmuno-S™ resin.

14. (canceled)

15. The method of claim 2, wherein said CEX membrane comprises Sartobind S™.

16. The method of claim 1, wherein the mixed mode resin is Capto Adhere™ resin.

17-20. (canceled)

21. The method of claim 1, wherein said Ab is loaded on to said CEX resin in a loading buffer comprising acetate ions and/or wherein said CEX buffer is equilibrated with an equilibration buffer comprising citrate ions.

22. (canceled)

23. The method of claim 21, wherein said equilibration buffer is devoid of acetate ions.

24. The method of claim 1, wherein the Ab is eluted from said CEX buffer using a citrate buffer.

25. The method of claim 1, wherein step (b) comprises washing said CEX resin with a citrate buffer and a HEPES buffer prior to eluting.

26-27. (canceled)

28. The method of claim 1, wherein said MM resin is equilibrated with an equilibration buffer comprising phosphate ions.

29. The method of claim 28, wherein said equilibration buffer is devoid of citrate ions.

30. The method of claim 1, wherein the Ab is contacted with said MM resin in a loading buffer comprising phosphate ions.

31. The method of claim 28, wherein said loading buffer further comprises citrate ions.

32. The method of claim 1, further comprising performing a viral inactivation step following step (c).

33. The method of claim 32, wherein said viral inactivation step is effected by lowering the pH of the mixture to a pH between 3 and 4.

34-45. (canceled)
